(19) United States
(12) Patent Application Publication
(10) Pub. No.: US 2023/0348568 A1
(43) Pub. Date: Nov. 2, 2023

(54) EPSTEIN-BARR VIRUS MONOCLONAL ANTIBODIES AND USES THEREOF

(71) Applicant: The U.S.A., as represented by the Secretary, Department of Health and Human Services, Bethesda, MD (US)

(72) Inventors: Jeffrey I. Cohen, Silver Spring, MD (US); Wei Bu, Potomac, MD (US); Nathan Board, Baltimore, MD (US); Kennichi Dowdell, Severna Park, MD (US)

(73) Assignee: The U.S.A., as represented by the Secretary, Department of Health and Human Services, Bethesda, MD (US)

(21) Appl. No.: 17/800,706

(22) PCT Filed: Feb. 19, 2021
(86) PCT No.: PCT/US2021/018836

§ 371 Date: Aug. 18, 2022

Related U.S. Application Data
(60) Provisional application No. 62/979,070, filed on Feb. 20, 2020.

(51) Int. Cl. C07K 16/08; C12N 15/63; G01N 33/569; A61P 31/20

(52) U.S. Cl. CPC C07K 16/085; C12N 15/63; G01N 33/56994; A61P 31/20; C07K 2317/565; C07K 2317/94; C07K 2317/31

(57) ABSTRACT

Monoclonal antibodies that specifically bind gp42 of Epstein-Barr virus (EBV) are described. The antibodies are capable of blocking fusion of EBV-infected cells and neutralizing EBV infection. Use of the EBV-specific monoclonal antibodies, and conjugates thereof, for the treatment and prophylaxis of EBV infection is also described.

### CROSS REFERENCE TO RELATED APPLICATIONS

This application claims the benefit of U.S. Provisional Application No. 62/979,070, filed Feb. 20, 2020, which is herein incorporated by reference in its entirety.

### ACKNOWLEDGMENT OF GOVERNMENT SUPPORT

This invention was made with government support under project number Z01 AI000978 awarded by the National Institutes of Health. The government has certain rights in the invention.

### FIELD

This disclosure concerns Epstein-Barr virus (EBV) monoclonal antibodies that block cell fusion and neutralize EBV infection of B cells. This disclosure further concerns use of the EBV-specific monoclonal antibodies, such as for the treatment or prophylaxis of EBV infection, including for the prevention of EBV-associated lymphoproliferative disease and other diseases.

### BACKGROUND

Epstein-Barr virus (EBV) is the most common cause of infectious mononucleosis and is associated with nearly 200,000 cancers and 140,000 deaths each year. EBV-associated cancers include Hodgkin's lymphoma, non-Hodgkin's lymphoma, Burkitt B cell lymphoma, and EBV post-transplant lymphoproliferative disease. The latent reservoir for EBV in the body is the B lymphocyte. Thus, blocking B cell infection is important for reducing EBV-related disease.

EBV can infect both B cells and epithelial cells; however, the method of entry differs between these two cell types. To initiate B cell infection, EBV glycoprotein 350 (gp350) binds to compliment receptor 2 (CR2; also known as CD21), followed by binding of gp42 to HLA class II molecules, which triggers fusion of EBV with the B cell, allowing virus entry into the cell. Fusion also requires the EBV proteins gH/gL, which are found complexed with gp42 as a heterotrimer, and gB. Infection of epithelial cells is initiated by the binding of the EBV protein BMRF2 to cellular integrins, followed by binding of gH/gL to ephrin receptor A2 and integrins, which triggers fusion by EBV gB.

### SUMMARY

Monoclonal antibodies that specifically bind EBV gp42 are disclosed. The gp42-specific antibodies are capable of neutralizing EBV infection and inhibiting fusion of EBV with B cells. The disclosed antibodies can be used, for example, for the treatment or prophylaxis of EBV infection, prevention of EBV-associated disease or infection in immunocompromised subjects, diagnosis of EBV infection, and detection of EBV in a biological sample.

Provided herein are monoclonal antibodies that specifically bind EBV gp42. In some embodiments, the monoclonal antibody includes the complementarity determining region (CDR) sequences of the A10 antibody or humanized versions of A10 disclosed herein. In some examples, the monoclonal antibody comprises the VH domain and/or VL domain of A10, A10⁻², A10⁻³, A10⁻⁴, A10⁻⁵, A10⁻⁶, A10⁻⁷, A10⁻⁸ or A10⁻⁹. In other embodiments, the monoclonal antibody includes the CDR sequences of the 4C12 antibody disclosed herein. In some examples, the monoclonal antibody comprises the VH domain and/or VL domain of 4C12.

In some embodiments, the monoclonal antibody is a mouse, non-human primate, humanized or chimeric antibody.

In some embodiments, the monoclonal antibody further includes a constant region. In some examples, the constant region includes at least one modification to increase the half-life, stability and/or function of the monoclonal antibody.

Conjugates, bispecific antibodies and fusion proteins that include a gp42-specific monoclonal antibody disclosed herein are also provided by the present disclosure.

Further provided are nucleic acid molecules and vectors encoding a gp42-specific monoclonal antibody disclosed herein.

Compositions that include a pharmaceutically acceptable carrier and a monoclonal antibody, conjugate, bispecific antibody, fusion protein, nucleic acid molecule or vector disclosed herein are also provided.

Also provided are methods of detecting EBV in a subject and methods of diagnosing a subject as having an EBV infection. In some embodiments, the methods include contacting a biological sample from the subject with a monoclonal antibody disclosed herein under conditions sufficient to form an immune complex; and detecting the presence of the immune complex in the sample. The presence of the immune complex in the sample detects EBV in the subject and/or diagnoses the subject as having an EBV infection.

Further provided are methods for the treatment or prophylaxis of an EBV infection in a subject. In some embodiments, the method includes administering to a subject a therapeutically effective amount of a monoclonal antibody, conjugate, bispecific antibody, fusion protein, nucleic acid molecule, vector or composition disclosed herein.

Further provided are methods for the prevention of EBV-associated lymphoproliferative disease in a subject, such as a subject who has or will receive a bone marrow or an organ transplant (such as a hand transplant, foot transplant, or a solid organ transplant, such as a liver transplant, kidney transplant, heart transplant, pancreas transplant, intestine transplant, or lung transplant), or a subject with a primary immunodeficiency disease (e.g., X-linked lymphoproliferative disease or MAGT1 deficiency). In some embodiments, the method includes administering to a subject a therapeutically effective amount of a monoclonal antibody, conjugate, bispecific antibody, fusion protein, nucleic acid molecule, vector or composition disclosed herein, for example soon after receiving the transplant, such as about 2 days, about 4 days, about 1 week, about 2 weeks, about 3 weeks, or about 4 weeks after the transplant. In some instances, the monoclonal antibody, conjugate, bispecific antibody, fusion protein, nucleic acid molecule, vector or composition is administered to a subject with an immunodeficiency, but not yet infected with EBV, to prevent or reduce disease associated with EBV infection.

Kits that include a monoclonal antibody, conjugate, bispecific antibody, fusion protein, nucleic acid molecule or vector disclosed herein; and buffer, one or more detection reagents, and/or instructional materials are further provided.

The foregoing and other objects and features of the disclosure will become more apparent from the following detailed description, which proceeds with reference to the accompanying figures.

### BRIEF DESCRIPTION OF THE DRAWINGS

FIGS. 1A-1B: Comparison of the ability of gp42-specific A10, 4C12 and F2-1 monoclonal antibodies to neutralize EBV infection in B cells. (FIG. 1A) Neutralization of EBV infection of B cells by mAbs A10, F2-1 and 4C12. (FIG. 1B) Comparison of B cell neutralization titers of mAbs A10, F2-1 and 4C12.

FIGS. 2A-2B: Comparison of the ability of the A10, 4C12 and F2-1 monoclonal antibodies to block EBV glycoprotein-mediated fusion in B cells. Inhibition of fusion of B cells by full-length (FL) mAbs (FIG. 2A) and Fabs (FIG. 2B). The percentage of fusion was defined as: (RLU [Rendla light units] of mAb Serum/RLU of Negative Serum)×100%. The dotted lines represent the background of the assay.

FIGS. 3A-3F: Binding affinity of the A10, F2-1 and 4C12 antibodies. Kinetics of binding of A10 Fab (FIG. 3A), F2-1 Fab (FIG. 3B) and 4C12 Fab (FIG. 3E) to gp42. Kinetics of binding of A10 Fab (FIG. 3C), F2-1 Fab (FIG. 3D) and 4C12 Fab (FIG. 3F) to gH/gL/gp42.

FIG. 4: gH/gL mAbs do not compete with A10 or F2-1 gp42 mAbs. Shown is a table of antibody cross-competition of EBV gp42 mAb A10 and F2-1 (primary antibodies) by gH/gL mAbs (A7, B10, C2, C5, F7, and F8) and gp42 mAbs (F2-1 and A10) (secondary antibodies). Antibody competition was measured by biolayer interferometry with recombinant gH/gL/gp42 protein.

FIGS. 5A-5C: gp42 mAb A10 competes with F2-1 mAb for binding to gp42 and gH/gL/gp42. Shown are tables of antibody cross-competition of mAb A10 by F2-1 and 4C12. Antibody competition was measured by biolayer interferometry with recombinant EBV type I gp42 (FIG. 5A), EBV type II gp42 (FIG. 5B) or gH/gL/gp42 (FIG. 5C) proteins.

FIG. 6: Humanized mice administered mAb A10 are protected from EBV viremia after intravenous challenge with EBV. Humanized mice (NOG mice engrafted with human CD34+ hematopoietic stem cells) were administered mAb A10 (or control mAb) intraperitoneally on days −1, 0, 1, 4, 7 and 10. Mice were given EBV (one million cell culture infectious doses) intravenously on day 0. EBV DNA copies were measured in blood at various weeks post infection (wpi). Animals that received mAb10 were protected from EBV viremia, while animals that received a control mAb were all infected.

FIGS. 7A-7B: A10 and F2-1 mAbs, but not 4C12 mAb, block gp42 binding to its receptor (HLA-DR). (FIG. 7A) Ni-NTA biosensors immobilized with gp42 or gH/gL/gp42 proteins were dipped into mixture of HLA-DR with A10, F2-1, 4C12, or control mAbs for 300 seconds for association by biolayer interferometry followed by dipping into buffer for dissociation. (FIG. 7B) The relative binding of gp42 or gH/gL/gp42 to HLA-DR in the presence of mAbs was calculated by Response/Response_{controlAb}×100.

FIGS. 8A-8B: A10 and F2-1 mAbs block gp42 and gH/gL/gp42 binding to human B cells. Fluorescent labeled gp42 (FIG. 8A) or gH/gL/gp42 (FIG. 8B) probes were mixed with anti-HLA, A10, F2-1, 4C12, or non-gp42 control mAbs followed by incubation with PBMCs from 4 individual healthy donors (data points on plots). The binding of probes on B cells was quantified by flow cytometry. The binding of probes only, without incubation of mAbs, was set as 100%.

FIG. 9: A10 mAb best protected humanized mice from EBV viremia after challenge. A10, 4C12 and F2-1 mAbs (0.5 mg per dose) were injected into humanized mice by I.P. injection on days −1, 0, 1, 4, 7, and 10. EBV (1×10⁶ Green Raji Units) was injected by I.V. injection on day 0. Blood was collected weekly and EBV copies were measured by real-time PCR.

FIG. 10: B cell neutralization titers of A10 mAb and humanized A10⁻² to A10⁻⁹ mAbs. Data shown are IC₅₀ titers with standard errors of the mean based on 3 independent experiments.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file, created on Feb. 19, 2021, 25.7 KB, which is incorporated by reference herein. In the accompanying sequence listing: SEQ ID NO: 1 is the nucleic acid sequence of the A10 VH domain.

SEQ ID NO: 2 is the amino acid sequence of the A10 VH domain

SEQ ID NO: 3 is the nucleic acid sequence of the A10 VL domain.

SEQ ID NO: 4 is the amino acid sequence of the A10 VL domain.

SEQ ID NO: 5 is the nucleic acid sequence of the 4C12 VH domain

SEQ ID NO: 6 is the amino acid sequence of the 4C12 VH domain.

SEQ ID NO: 7 is the nucleic acid sequence of the 4C12 VL domain.

SEQ ID NO: 8 is the amino acid sequence of the 4C12 VL domain

SEQ ID NO: 9 is an amino acid sequence of EBV gp42.

SEQ ID NO: 10 is the amino acid sequence of the A10⁻² VH domain.

SEQ ID NO: 11 is the amino acid sequence of the A10⁻² VL domain.

SEQ ID NO: 12 is the amino acid sequence of the A10⁻³ VH domain.

SEQ ID NO: 13 is the amino acid sequence of the A10⁻³ VL domain.

SEQ ID NO: 14 is the amino acid sequence of the A10⁻⁴ VH domain.

SEQ ID NO: 15 is the amino acid sequence of the A10⁻⁴ VL domain.

SEQ ID NO: 16 is the amino acid sequence of the A10⁻⁵ VH domain.

SEQ ID NO: 17 is the amino acid sequence of the A10⁻⁵ VL domain.

SEQ ID NO: 18 is the amino acid sequence of the A10⁻⁶ VH domain.

SEQ ID NO: 19 is the amino acid sequence of the A10⁻⁶ VL domain.

SEQ ID NO: 20 is the amino acid sequence of the A10⁻⁷ VH domain.

SEQ ID NO: 21 is the amino acid sequence of the A10⁻⁷ VL domain.

SEQ ID NO: 22 is the amino acid sequence of the A10⁻⁸ VH domain.

SEQ ID NO: 23 is the amino acid sequence of the A10⁻⁸ VL domain.

SEQ ID NO: 24 is the amino acid sequence of the A10⁻⁹ VH domain.

SEQ ID NO: 25 is the amino acid sequence of the A10⁻⁹ VL domain.

### DETAILED DESCRIPTION

### I. Abbreviations

ADCC antibody-dependent cell-mediated cytotoxicity
CDC complement-dependent cytotoxicity
CDR complementarity determining region
EBV Epstein-Barr virus
gp42 glycoprotein 42
gp350 glycoprotein 350
gH/gL glycoprotein H and glycoprotein L
gB glycoprotein B
mAb monoclonal antibody
RLU Renilla light unit
VH variable heavy
VL variable light

### II. Terms and Methods

Unless otherwise noted, technical terms are used according to conventional usage. Definitions of common terms in molecular biology may be found in Benjamin Lewin, Genes X, published by Jones & Bartlett Publishers, 2009; and Meyers et al. (eds.), The Encyclopedia of Cell Biology and Molecular Medicine, published by Wiley-VCH in 16 volumes, 2008; and other similar references.

As used herein, the singular forms “a,” “an,” and “the,” refer to both the singular as well as plural, unless the context clearly indicates otherwise. For example, the term “an antigen” includes single or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various embodiments, the following explanations of terms are provided:

Administration: The introduction of a composition, such as an antibody provided herein, into a subject by a chosen route. Administration can be local or systemic. For example, if the chosen route is intravenous, the composition is administered by introducing the composition into a vein of the subject. Exemplary routes of administration include, but are not limited to, oral, injection (such as subcutaneous, intramuscular, intradermal, intraperitoneal, intraosseous, and intravenous), sublingual, rectal, transdermal (for example, topical), intranasal, vaginal, and inhalation routes.

Antibody: A polypeptide ligand comprising at least one variable region that recognizes and binds (such as specifically recognizes and specifically binds) an epitope of an antigen. Mammalian immunoglobulin molecules are composed of a heavy (H) chain and a light (L) chain, each of which has a variable region, termed the variable heavy (VH) region and the variable light (VL) region, respectively. Together, the VH region and the VL region are responsible for binding the antigen recognized by the antibody. There are five main heavy chain classes (or isotypes) of mammalian immunoglobulin, which determine the functional activity of an antibody molecule: IgM, IgD, IgG, IgA and IgE. Antibody isotypes not found in mammals include IgX, IgY, IgW and IgNAR. IgY is the primary antibody produced by birds and reptiles, and is functionally similar to mammalian IgG and IgE. IgW and IgNAR antibodies are produced by cartilaginous fish, while IgX antibodies are found in amphibians.

Antibody variable regions contain “framework” regions and hypervariable regions, known as “complementarity determining regions” or “CDRs.” The CDRs are primarily responsible for binding to an epitope of an antigen. The framework regions of an antibody serve to position and align the CDRs in three-dimensional space. The amino acid sequence boundaries of a given CDR can be readily determined using any of a number of well-known numbering schemes, including those described by Kabat et al. (Sequences of Proteins of Immunological Interest, U.S. Department of Health and Human Services, 1991; the “Kabat” numbering scheme), Chothia et al. (see Chothia and Lesk, J Mol Biol 196:901-917, 1987; Chothia et al., Nature 342:877, 1989; and Al-Lazikani et al., JMB 273,927-948, 1997; the “Chothia” numbering scheme), Kunik et al. (see Kunik et al., PLoS Comput Biol 8:e1002388, 2012; and Kunik et al., Nucleic Acids Res 40(Web Server issue):W521-524, 2012; “Paratome CDRs”) and the ImMunoGeneTics (IMGT) database (see, Lefranc, Nucleic Acids Res 29:207-9, 2001; the “IMGT” numbering scheme). The Kabat, Paratome and IMGT databases are maintained online.

A “single-domain antibody” refers to an antibody having a single domain (a variable domain) that is capable of specifically binding an antigen, or an epitope of an antigen, in the absence of an additional antibody domain. Single-domain antibodies include, for example, V_{H} domain antibodies, V_{NAR} antibodies, camelid V_{H}H antibodies, and V_{L} domain antibodies. V_{NAR} antibodies are produced by cartilaginous fish, such as nurse sharks, wobbegong sharks, spiny dogfish and bamboo sharks. Camelid V_{H}H antibodies are produced by several species including camel, llama, alpaca, dromedary, and guanaco, which produce heavy chain antibodies that are naturally devoid of light chains.

A “monoclonal antibody” is an antibody produced by a single clone of lymphocytes or by a cell into which the coding sequence of a single antibody has been transfected. Monoclonal antibodies are produced by methods known to those of skill in the art. Monoclonal antibodies include humanized monoclonal antibodies.

A “chimeric antibody” has framework residues from one species, such as human, and CDRs (which generally confer antigen binding) from another species.

A “humanized” antibody is an immunoglobulin including a human framework region and one or more CDRs from a non-human (for example a mouse, rabbit, rat, shark or synthetic) immunoglobulin. The non-human immunoglobulin providing the CDRs is termed a “donor,” and the human immunoglobulin providing the framework is termed an “acceptor.” In one embodiment, all CDRs are from the donor immunoglobulin in a humanized immunoglobulin. Constant regions need not be present, but if they are, they must be substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. A humanized antibody binds to the same antigen as the donor antibody that provides the CDRs. Humanized or other monoclonal antibodies can have additional conservative amino acid substitutions which have substantially no effect on antigen binding or other immunoglobulin functions.

Binding affinity: Affinity of an antibody for an antigen, such as an antibody provided herein that binds gp42. In one embodiment, affinity is calculated by a modification of the Scatchard method described by Frankel et al., Mol. Immunol., 16:101-106, 1979. In another embodiment, binding affinity is measured by an antigen/antibody dissociation rate. In another embodiment, a high binding affinity is measured by a competition radioimmunoassay. In another embodiment, binding affinity is measured by ELISA. In other embodiments, antibody affinity is measured by flow cytometry or by surface plasmon reference. An antibody that “specifically binds” an antigen (such as EBV gp42) is an antibody that binds the antigen with high affinity and does not significantly bind other unrelated antigens.

In some examples, an antibody or fragment thereof (such as an anti-gp42 antibody provided herein) specifically binds to a target (such as gp42) with a binding constant that is at least 10³ M⁻¹ greater, 10⁴ M⁻¹ greater or 10⁵ M⁻¹ greater than a binding constant for other molecules in a sample or subject. In some examples, an antibody (e.g., monoclonal antibody) or fragments thereof, has an equilibrium constant (Kd) of 10 nM or less, such as 5 nM or less, 2.5 nM or less, 2 nM or less, 1.5 nM or less, 1 nM or less, 0.9 nM or less, 0.8 nM or less, 0.7 nM or less, 0.6 nM or less, 0.5 nM or less, 0.4 nM or less, 0.3 nM or less, 0.2 nM or less or 0.1 nM or less. For example, an antibody or fragment thereof binds to a target, such as gp42 with a binding affinity of at least about 10×10⁻¹⁰ M, at least about 9×10⁻¹⁰ M, at least about 8×10⁻¹⁰ M, at least about 7×10⁻¹⁰ M, at least about 6×10⁻¹⁰ M, at least about 5×10⁻¹⁰ M at least about 2.5×10⁻¹° M, at least about 2×10⁻¹⁰ M, at least about 1×10⁻¹⁰, at least about 0.75×10⁻¹⁰, at least about 0.5×10⁻¹⁰, at least about 0.4×10⁻¹⁰, at least about 0.3×10⁻¹⁰, at least about 0.2×10⁻¹⁰ M, at least about 0.1×10⁻¹⁰ M, at least about 0.05×10⁻¹⁰ M, or at least about 0.01×10⁻¹⁰ M. In certain embodiments, a specific binding agent (such as an anti-gp42 antibody provided herein) that binds to its target (such as gp42) has a dissociation constant (Kd) of ≤10 nM, ≤5 nM, ≤4 nM, ≤3 nM, ≤2 nM, ≤1.5 nM, ≤1 nM, ≤0.9 nM, ≤0.8 nM, ≤0.7 nM, ≤0.6 nM, ≤0.5 nM, ≤0.4 nM, ≤0.3 nM, ≤0.2 nM, ≤0.1 nM, ≤0.05 nM, or ≤0.01 nM (e.g., 10⁻⁹M or less, e.g., from 10⁻⁹M to 10⁻¹³M, e.g., from 10⁻¹⁰ M to 10⁻¹¹ M). In one embodiment, Kd is measured by a radiolabeled antigen binding assay (RIA) performed with the Fab version of an antibody of interest and its antigen (see, e.g., Chen et al., J. Mol. Biol. 293:865-881, 1999). In another example, Kd is measured using surface plasmon resonance assays using a BIACORES-2000 or a BIACORES-3000 (BIAcore, Inc., Piscataway, N.J.) at 25° C. with immobilized antigen CMS chips at about 10 response units (RU).

Bispecific antibody: A recombinant protein that includes antigen-binding fragments of two different monoclonal antibodies and is thereby capable of binding two different antigens, or two different epitopes on the same antigen. In some embodiments herein, the bispecific antibody targets both EBV gp42 and another EBV protein, such as gp350, gH/gL or gB. In other embodiments, both antigen-binding fragments of the bispecific antibody bind gp42, but bind different epitopes of gp42. Similarly, a multi-specific antibody is a recombinant protein that includes antigen-binding fragments of at least two different monoclonal antibodies, such as two, three or four different monoclonal antibodies.

Complementarity determining region (CDR): A region of hypervariable amino acid sequence that defines the binding affinity and specificity of an antibody. The light and heavy chains of a mammalian immunoglobulin each have three CDRs, designated L-CDR1, L-CDR2, L-CDR3 and H-CDR1, H-CDR2, H-CDR3, respectively. A single-domain antibody contains three CDRs, referred to herein as CDR1, CDR2 and CDR3.

Conjugate: In the context of the present disclosure, a “conjugate” is an antibody or antibody fragment (such as an antigen-binding fragment) covalently linked to an effector molecule or a second protein (such as a second antibody). The effector molecule can be, for example, a drug, toxin, therapeutic agent, detectable label, protein, nucleic acid, lipid, nanoparticle, photon absorber, carbohydrate or recombinant virus.

Conservative variant: A protein containing conservative amino acid substitutions that do not substantially affect or decrease the affinity of a protein, such as an antibody to EBV gp42. For example, a monoclonal antibody that specifically binds gp42 can include at most about 1, at most about 2, at most about 5, at most about 10, or at most about 15 conservative substitutions (for example, 1, 2, 3, 4, 5, 6, 7, 8, 9, 10, 11, 12, 13, 14 or 15 conservative substitutions) and specifically bind the gp42 polypeptide. The term “conservative variant” also includes the use of a substituted amino acid in place of an unsubstituted parent amino acid, provided that the antibody specifically binds gp42. Non-conservative substitutions are those that reduce an activity or binding to gp42.

Conservative amino acid substitution tables providing functionally similar amino acids are known. The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Contacting: Placement in direct physical association; includes both in solid and liquid form.

Diagnostic: Identifying the presence or nature of a pathologic condition, such as an EBV infection. Diagnostic methods differ in their sensitivity and specificity. The “sensitivity” of a diagnostic assay is the percentage of diseased individuals who test positive (percent of true positives). The “specificity” of a diagnostic assay is one minus the false positive rate, where the false positive rate is defined as the proportion of those without the disease who test positive. While a particular diagnostic method may not provide a definitive diagnosis of a condition, it suffices if the method provides a positive indication that aids in diagnosis. “Prognostic” is the probability of development (such as severity) of a pathologic condition.

Effector molecule: The portion of a chimeric molecule that is intended to have a desired effect on a cell to which the chimeric molecule is targeted. Effector molecule is also known as an effector moiety (EM), therapeutic agent, diagnostic agent, or similar terms. Therapeutic agents (or drugs) include such compounds as nucleic acids, proteins, peptides, amino acids or derivatives, glycoproteins, radioisotopes, photon absorbers, lipids, carbohydrates, or recombinant viruses. Nucleic acid therapeutic and diagnostic moieties include antisense nucleic acids, derivatized oligonucleotides for covalent cross-linking with single or duplex DNA, and triplex forming oligonucleotides. Alternatively, the molecule linked to a targeting moiety, such as an anti-gp42 antibody, may be an encapsulation system, such as a liposome or micelle that contains a therapeutic composition such as a drug, a nucleic acid (such as an antisense nucleic acid), or another therapeutic moiety that can be shielded from direct exposure to the circulatory system. Means of preparing liposomes attached to antibodies are known (see, for example, U.S. Pat. No. 4,957,735; and Connor et al., Pharm Ther 28:341-365, 1985). Diagnostic agents or moieties include radioisotopes and other detectable labels. Detectable labels useful for such purposes are also known, and include radioactive isotopes such as ³⁵S, ¹¹C, ¹³N, ¹⁵O, ¹⁸F, ¹⁹F, ^{99m}Tc, ¹³¹I, ³H, ¹⁴C, ¹⁵N, ⁹⁰Y, ⁹⁹Tc, ¹¹¹In and ¹²⁵I, fluorophores, chemiluminescent agents, and enzymes.

Epitope: An antigenic determinant. These are particular chemical groups or peptide sequences on a molecule that are antigenic (that elicit a specific immune response). An antibody specifically binds a particular antigenic epitope on a polypeptide, such as EBV gp42.

Epstein-Barr virus (EBV): A member of the herpes virus family EBV is also known as human herpesvirus 4 (HHV4). The genome of EBV is comprised of double-stranded DNA of approximately 172 kb, which is surrounded by a nucleocapsid, a protein tegument and a lipid envelope. There are two types of EBV, type 1 and type 2, which differ in their transforming and reactivation ability. EBV primarily infects B lymphocytes and epithelial cells. Infection with EBV can cause infectious mononucleosis, which is characterized by, for example, fatigue, fever, swollen lymph nodes, inflamed throat, enlarged spleen, swollen liver and rash. EBV is also associated with several non-malignant and malignant diseases and multiple sclerosis. EBV is most often spread via bodily fluids, including saliva, blood and semen.

Framework region: Amino acid sequences interposed between CDRs. Framework regions of an immunoglobulin molecule include variable light and variable heavy framework regions.

Fusion protein: A protein comprising at least a portion of two different (heterologous) proteins.

Glycoprotein 42 (gp42): An EBV protein that is required for infection of B cells and for mediating B cell fusion Amino acid sequences for EBV gp42 are publicly available, such as GenBank Accession No. P03205.1, deposited Jul. 21, 1986, which is set forth herein as SEQ ID NO: 9. The soluble form of gp42 is comprised of amino acids 34-223 of SEQ ID NO: 9.

Glycoprotein H (gH): An EBV protein that plays a role in membrane fusion and virus entry. gH is a membrane-bound glycoprotein that forms a heterodimer with gL. Amino acid sequences of EBV gH are publicly available, such as GenBank Accession No. P03231.1, deposited Jul. 21, 1986.

Glycoprotein L (gL): An EBV protein that plays a role in membrane fusion and virus entry. gL is a soluble glycoprotein that forms a heterodimer with gH. Amino acid sequences of EBV gL are publicly available, such as GenBank Accession No. P03212.1, deposited Jul. 21, 1986.

Heterologous: Originating from a separate genetic source or species.

Immune response: A response of a cell of the immune system, such as a B cell, T cell, or monocyte, to a stimulus. In one embodiment, the response is specific for a particular antigen (an “antigen-specific response”). In one embodiment, an immune response is a T cell response, such as a CD4⁺ response or a CD8⁺ response. In another embodiment, the response is a B cell response, and results in the production of specific antibodies.

Immunocompromised: Having a weakened immune response as a result of a genetic disorder (immunodeficiency), viral infection, malnutrition or due to treatment with immunosuppressive drugs.

Immunosuppressed: A state in which the immune system is suppressed by pharmaceutical treatment, such as during the treatment of particular diseases or following organ transplantation.

Isolated: An “isolated” biological component, such as a nucleic acid molecule, protein (including antibodies) or organelle, has been substantially separated or purified away from other biological components in the environment (such as a cell) in which the component occurs, for example other chromosomal and extra-chromosomal DNA and RNA, proteins and organelles. Nucleic acids and proteins that have been “isolated” include nucleic acid molecules and proteins purified by standard purification methods. The term also embraces nucleic acid molecules and proteins prepared by recombinant expression in a host cell as well as chemically synthesized nucleic acid molecules and proteins.

Label: A detectable compound or composition that is conjugated directly or indirectly to another molecule, such as an antibody or a protein, to facilitate detection of that molecule. Specific, non-limiting examples of labels include fluorescent tags, enzymatic linkages, and radioactive isotopes. In one example, a “labeled antibody” refers to incorporation of another molecule in the antibody. For example, the label is a detectable marker, such as the incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Various methods of labeling polypeptides and glycoproteins are known and may be used. Examples of labels for polypeptides include, but are not limited to, the following: radioisotopes or radionucleotides (such as ³⁵S, ¹¹C, ¹³N, ¹⁵O, ¹⁸F, ¹⁹F, ^{99m}Tc, ¹³¹I, ³H, ¹⁴C, ¹⁵N, ⁹⁰Y, ⁹⁹Tc, ¹¹¹In and ¹²⁵I), fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, predetermined polypeptide epitopes recognized by a secondary reporter (such as a leucine zipper pair sequences, binding sites for secondary antibodies, metal binding domains, epitope tags), or magnetic agents, such as gadolinium chelates. In some embodiments, labels are attached by spacer arms of various lengths to reduce potential steric hindrance.

Linker: In some cases, a linker is a peptide within an antibody binding fragment (such as an Fv fragment) which serves to indirectly bond the variable heavy chain to the variable light chain. “Linker” can also refer to a peptide serving to link a targeting moiety, such as an antibody, to an effector molecule, such as a cytotoxin or a detectable label. The terms “conjugating,” “joining,” “bonding” or “linking” refer to making two polypeptides into one contiguous polypeptide molecule, or to covalently attaching a radionuclide or other molecule to a polypeptide, such as an antibody. The linkage can be either by chemical or recombinant means. “Chemical means” refers to a reaction between the antibody moiety and the effector molecule such that there is a covalent bond formed between the two molecules to form one molecule.

Lymphoproliferative disease or disorder: A disease or disorder characterized by the uncontrolled or abnormal proliferation of lymphocytes. Lymphoproliferative diseases typically occur in immunocompromised or immunosuppressed subjects. Examples of lymphoproliferative diseases/disorders include, but are not limited to, lymphomas (e.g. B cell lymphoma, T cell lymphoma, follicular lymphoma), leukemias (e.g. chronic lymphocytic leukemia, acute lymphoblastic leukemia, hairy cell leukemia), multiple myeloma, post-transplant lymphoproliferative disorder, autoimmune lymphoproliferative syndrome, EBV-associated lymphoproliferative disease and Castleman disease.

Modification: A change in a nucleic acid or protein sequence. For example, amino acid sequence modifications include, for example, substitutions, insertions and deletions, or combinations thereof. Insertions include amino and/or carboxyl terminal fusions as well as intrasequence insertions of single or multiple amino acid residues. Deletions are characterized by the removal of one or more amino acid residues from the protein sequence. Substitutional modifications are those in which at least one residue has been removed and a different residue inserted in its place. Amino acid substitutions are typically of single residues, but can occur at a number of different locations at once. In some embodiments herein, the modification (such as a substitution, insertion or deletion) of an antibody constant region results in a change in function, such as an increase in binding to Fc receptors, or an increase in half-life, stability and/or function of the antibody. Protein (such as antibody) modifications can be prepared by modification of nucleotides in the DNA encoding the protein, thereby producing DNA encoding the modification. A “modified” protein or nucleic acid is one that has one or more modifications as outlined above.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Pharmaceutically acceptable carriers: The pharmaceutically acceptable carriers of use are conventional. Remington: The Science and Practice of Pharmacy, The University of the Sciences in Philadelphia, Editor, Lippincott, Williams, & Wilkins, Philadelphia, PA, 21^{st} Edition (2005), describes compositions and formulations suitable for pharmaceutical delivery of the antibodies and other compositions disclosed herein. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Preventing, treating or ameliorating a disease: Inhibiting the full development of a disease or condition, for example, in a subject who is at risk of or has an EBV infection. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the viral load, an improvement in the overall health or well-being of the subject, or by other parameters that are specific to the particular disease. A “prophylactic” treatment is a treatment administered to a subject who does not exhibit signs of a disease for the purpose of reducing the risk of developing pathology.

The term “reduces” is a relative term, such that an agent reduces a disease or condition (or a symptom of a disease or condition) if the disease or condition is quantitatively diminished following administration of the agent, or if it is diminished following administration of the agent, as compared to a reference agent. Similarly, the term “prevents” does not necessarily mean that an agent completely eliminates the disease or condition, so long as at least one characteristic of the disease or condition is eliminated. Thus, an antibody that reduces or prevents an infection, can, but does not necessarily completely, eliminate such an infection, so long as the infection is measurably diminished, for example, by at least about 50%, such as by at least about 70%, or about 80%, or even by about 90% the infection in the absence of the agent, or in comparison to a reference agent.

Purified: The term purified does not require absolute purity; rather, it is intended as a relative term. Thus, for example, a purified peptide preparation is one in which the peptide or protein (such as an antibody) is more enriched than the peptide or protein is in its natural environment within a cell. In one embodiment, a preparation is purified such that the protein or peptide represents at least 50% of the total peptide or protein content of the preparation. Substantial purification denotes purification from other proteins or cellular components. A substantially purified protein is at least 60%, 70%, 80%, 90%, 95% or 98% pure. Thus, in one specific, non-limiting example, a substantially purified protein is 90% free of other proteins or cellular components.

Recombinant: A recombinant nucleic acid or protein is one that has a sequence that is not naturally occurring or has a sequence that is made by an artificial combination of two otherwise separated segments of sequence. This artificial combination is often accomplished by chemical synthesis or by the artificial manipulation of isolated segments of nucleic acids, for example, by genetic engineering techniques.

Sample (or biological sample): A biological specimen containing genomic DNA, RNA (including mRNA), protein, or combinations thereof, obtained from a subject. Examples include, but are not limited to, peripheral blood, serum, plasma, tissue (such as lymphoid tissue), cells (such as lymphocytes, particularly B lymphocytes), urine, saliva, sputum, cerebral spinal fluid, tissue biopsy, fine needle aspirate, surgical specimen, and autopsy material.

Sequence identity: The similarity between amino acid or nucleic acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide or nucleic acid molecule will possess a relatively high degree of sequence identity when aligned using standard methods.

Methods of alignment of sequences for comparison are well known. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, MD) and on the internet, for use in connection with the sequence analysis programs blastp, blastn, blastx, tblastn and tblastx. A description of how to determine sequence identity using this program is available on the NCBI website on the internet.

Homologs and variants of an antibody that specifically binds an EBV gp42 polypeptide are typically characterized by possession of at least about 75%, for example at least about 80%, 90%, 95%, 96%, 97%, 98% or 99% sequence identity counted over the full-length alignment with the amino acid sequence of the antibody using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10⁻²⁰ amino acids, and may possess sequence identities of at least 85% or at least 90% or 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Subject: Living multi-cellular vertebrate organisms, a category that includes both human and veterinary subjects, including human and non-human mammals.

Synthetic: Produced by artificial means in a laboratory, for example a synthetic nucleic acid or protein (for example, an antibody) can be chemically synthesized in a laboratory.

Therapeutically effective amount: A quantity of a specific substance sufficient to achieve a desired effect in a subject being treated. For instance, this can be the amount necessary to inhibit or suppress virus replication and/or spread. In one embodiment, a therapeutically effective amount is the amount necessary to eliminate or reduce titer of EBV, such as a reduction of at least 10%, at least 20%, at least 30%, at least 40%, at least 50%, at least 75%, at least 80%, at least 90%, at least 95%, or even 100%, as compared to a EBV titer without the treatment. When administered to a subject, a dosage will generally be used that will achieve concentrations that have been shown to achieve a desired in vitro effect.

Vector: A nucleic acid molecule as introduced into a host cell, thereby producing a transformed host cell. A vector may include nucleic acid sequences that permit it to replicate in a host cell, such as an origin of replication. A vector may also include one or more selectable marker genes and other genetic elements known in the art. In some embodiments, the vector is a virus vector, such as a lentivirus vector or an adeno-associated viral vector (AAV). In another example, a vector is a plasmid.

### III. Monoclonal Antibodies Specific for EBV Glycoprotein 42

Described herein are monoclonal antibodies that bind EBV gp42 with high affinity. Antibody A10 is a cynomolgus monkey antibody and antibody 4C12 is a mouse antibody. It is disclosed herein that both A10 and 4C12 specifically bind gp42 and are capable of inhibiting fusion of EBV-infected B cells. Antibody A10 also potently neutralizes EBV infection of B cells and protects against viremia in a humanized mouse model of EBV infection. The nucleotide and amino acid sequences of A10 and 4C12 are provided below and are set forth herein as SEQ ID NOs: 1-8. The location of each CDR, as determined by IMGT, for each of the antibodies is also shown below (underlined). Other numbering schemes, such as Chothia or Kabat, can also be used to determine the boundaries of each CDR. Eight humanized versions of A10 are further disclosed herein (A10⁻² to A10⁻⁹, set forth as SEQ ID NOs: 10⁻²⁵).

A10 VH nucleotide sequence
(SEQ ID NO: 1)
CAGGTGCAGCTGCAGGAGTCAGGTCCAGGACTGGTGAAGCCCTCACAGAC
CCTGTCACTCACCTGTGCCATCTCCGGGGACAGTGTCTCTAGCAACAGTG
CTACTTGGAACTGGATCAGGCAGTCGCCATCGAGAGGCCTTGAGTGGCTG
GGAAGGACATACTACAGGTCCAAGTGGTATTATGATTATGCACAATCTGT
GCAAAATCGAATAAGCATCAACCCAGACACATCCAAGAACCAGTTCTCCC
TGCAGCTGAACTCTGTGACCCCCGAGGACATGGCCGTGTATTACTGTGCA
AGAGGGGATTTTGATGCTTTTGATTTCTGGGGCCAAGGGCTCAGGGTCAC
CGTCTCTTCA
A10 VH amino acid sequence
(SEQ ID NO: 2)
QVQLQESGPGLVKPSQTLSLTCAISGDSVSSNSATWNWIRQSPSRGLEWL
GRTYYRSKWYYDYAQSVQNRISINPDTSKNQFSLQLNSVTPEDMAVYYCA
RGDFDAFDFWGQGLRVTVSS
A10 VL nucleotide sequence
(SEQ ID NO: 3)
TCGTATGAGCTGACTCAGCCACCCTCAGTGTCAGCGGCCTCAGGACAGAC
GGCCAGGATCACCTGTGGGGGAGACAACATTGGAAGTAAAAATGTGCAGT
GGTACCAGCAGAAGCCAGCGCAGGCCCCTGTGCTGGTCATCTATGCTGAT
AGTAAACGGCCCTCAGGGGTCCCTGAGCGATTCTCTGGCTCCAACTCAGG
GAACACGGCCACCCTGACCATCAGCGGGGTCGAGGCCGGGGATGAGGCTG
ACTATTACTGTCAGGTGTGGGACAGGAGTAGTAACATCTTCGGTGTTGGG
ACCCGGCTCACCGTCCTAGGT
A10 VL amino acid sequence
(SEQ ID NO: 4)
SYELTQPPSVSAASGQTARITCGGDNIGSKNVQWYQQKPAQAPVLVIYAD
SKRPSGVPERFSGSNSGNTATLTISGVEAGDEADYYCQVWDRSSNIFGVG
TRLTVLG
Humanized A10-2 VH amino acid sequence
(SEQ ID NO: 10)
QVQLQESGPGLVKPSQTLSLTCTISGDSVSSNSATWNWIRQPPGKGLEWL
GRTYYRSKWYYDYAQSLKSRVTISPDTSKNQFSLKLSSVTAADTAVYYCA
RGDFDAFDFWGQGTLVTVSS
Humanized A10-2 VL amino acid sequence
(SEQ ID NO: 11)
SYELMQPPSVSVSPGQTARITCSGDNIGSKNVQWYQQKPGQAPVLVIYAD
SKRPSGIPERFSGSNSGNTATLTISGVQAEDEADYYCQVWDRSSNIFGGG
TKLTVLG
Humanized A10-3 VH amino acid sequence
(SEQ ID NO: 12)
QVQLQESGPGLVKPSQTLSLTCTISGDSVSSNSATWNWIRQPPGKGLEWL
GRTYYRSKWYYDYAQSLKSRVTISPDTSKNQFSLKLSSVTAADTAVYYCA
RGDFDAFDFWGQGTLVTVSS
Humanized A10-3 VL amino acid sequence
(SEQ ID NO: 13)
SYELTQPPSVSVSPGQTARITCSGDNIGSKNVQWYQQKSGQAPVLVIYAD
SKRPSGIPERFSGSNSGNMATLTISGAQVEDEDDYYCQVWDRSSNIFGGG
TKLTVLG
Humanized A10-4 VH amino acid sequence
(SEQ ID NO: 14)
QVQLQESGPGLVKPSQTLSLTCTISGDSVSSNSATWNWIRQPPGKGLEWL
GRTYYRSKWYYDYAQSLKSRVTISPDTSKNQFSLKLSSVTAADTAVYYCA
RGDFDAFDFWGQGTLVTVSS
Humanized A10-4 VL amino acid sequence
(SEQ ID NO: 15)
SYVLTQPPSVSAAPGQKVTISCSGDNIGSKNVQWYQQLPGTAPKLLIYAD
SKRPSGIPDRFSGSNSGNSATLGITGLQTGDEADYYCQVWDRSSNIFGGG
TKLTVLG
Humanized A10-5 VH amino acid sequence
(SEQ ID NO: 16)
QVQLQESGPGLVKPSQTLSLTCTISGDSVSSNSATWNWIRQPPGKGLEWL
GRTYYRSKWYYDYAQSLKSRVTISPDTSKNQFSLKLSSVTAADTAVYYCA
RGDFDAFDFWGQGTLVTVSS
Humanized A10-5 VL amino acid sequence
(SEQ ID NO: 17)
QYVLTQPPSVSAAPGQKVTISCSGDNIGSKNVQWYQQLPGTAPKLLIYAD
SKRPSGIPDRFSGSNSGNSATLGITGLQTGDEADYYCQVWDRSSNIFGGG
TKLTVLG
Humanized A10-6 VH amino acid sequence
(SEQ ID NO: 18)
QVQLQESGPGLVKPSQTLSLTCTISGDSVSSNSATWNWIRQPPGKGLEWI
GRTYYRSKWYYDYAQSLKSRVTISPDTSKNQFSLKLSSVTAADTAVYYCA
RGDFDAFDFWGQGTLVTVSS
Humanized A10-6 VL amino acid sequence
(SEQ ID NO: 19)
SYELMQPPSVSVSPGQTARITCSGDNIGSKNVQWYQQKPGQAPVLVIYAD
SKRPSGIPERFSGSNSGNTATLTISGVQAEDEADYYCQVWDRSSNIFGGG
TKLTVLG
Humanized A10-7 VH amino acid sequence
(SEQ ID NO: 20)
QVQLQESGPGLVKPSQTLSLTCTISGDSVSSNSATWNWIRQPPGKGLEWI
GRTYYRSKWYYDYAQSLKSRVTISPDTSKNQFSLKLSSVTAADTAVYYCA
RGDFDAFDFWGQGTLVTVSS
Humanized A10-7 VL amino acid sequence
(SEQ ID NO: 21)
SYELTQPPSVSVSPGQTARITCSGDNIGSKNVQWYQQKSGQAPVLVIYAD
SKRPSGIPERFSGSNSGNMATLTISGAQVEDEDDYYCQVWDRSSNIFGGG
TKLTVLG
Humanized A10-8 VH amino acid sequence
(SEQ ID NO: 22)
QVQLQESGPGLVKPSQTLSLTCTISGDSVSSNSATWNWIRQPPGKGLEWI
GRTYYRSKWYYDYAQSLKSRVTISPDTSKNQFSLKLSSVTAADTAVYYCA
RGDFDAFDFWGQGTLVTVSS
Humanized A10-8 VL amino acid sequence
(SEQ ID NO: 23)
SYVLTQPPSVSAAPGQKVTISCSGDNIGSKNVQWYQQLPGTAPKLLIYAD
SKRPSGIPDRFSGSNSGNSATLGITGLQTGDEADYYCQVWDRSSNIFGGG
TKLTVLG
Humanized A10-9 VH amino acid sequence
(SEQ ID NO: 24)
QVQLQESGPGLVKPSQTLSLTCTISGDSVSSNSATWNWIRQPPGKGLEWI
GRTYYRSKWYYDYAQSLKSRVTISPDTSKNQFSLKLSSVTAADTAVYYCA
RGDFDAFDFWGQGTLVTVSS
Humanized A10-9 VL amino acid sequence
(SEQ ID NO: 25)
QYVLTQPPSVSAAPGQKVTISCSGDNIGSKNVQWYQQLPGTAPKLLIYAD
SKRPSGIPDRFSGSNSGNSATLGITGLQTGDEADYYCQVWDRSSNIFGGG
TKLTVLG

| | CDR1 | CDR2 | CDR3 |
|---|---|---|---|
| | residues | residues | residues |
| Monkey and humanized A10 VH domains | 26-35 | 53-61 | 100-109 |
| (SEQ ID NOs: 2, 10, 12, 14, 16, 18, | | | |
| 20, 22 and 24) | | | |
| Monkey and humanized A10 VL domains | 26-31 | 49-51 | 88-99 |
| (SEQ ID NOs: 4, 11, 13, 15, 17, 19, | | | |
| 21, 23 and 25) | | | |

4C12 VH nucleotide sequence
(SEQ ID NO: 5)
GAGGTCCAGCTTCAGCAGTCAGGACCTGAGCTGGTGAAACCTGGGGCCTC
AGTGAAGATATCCTGCAAGGCTTCTGGATACACATTCACTGACTACAACA
TGCACTGGGTGAAGCAGAGCCATGGAAAGAACCTTGAGTGGATTGGATTT
ATTTATCTTTACAATGGTGGTACTGGCTACAACCAGAACTTCAAGAGCAA
GGCCACATTGACTGTAGACAATTCCTCCAGCACAGCCTACATGGAGCTCC
GCAGCCTGACATCTGAGGACTCTGCAGTCTTTTACTGTGCAAGGGACTAC
TATGGTAACCCCTATGCTATGGACTACTGGGGTCAAGGAACCTCAGTCAC
CGTCTCCTCA
4C12 VH amino acid sequence
(SEQ ID NO: 6)
EVQLQQSGPELVKPGASVKISCKASGYTFTDYNMHWVKQSHGKNLEWIGF
IYLYNGGTGYNQNFKSKATLTVDNSSSTAYMELRSLTSEDSAVFYCARDY
YGNPYAMDYWGQGTSVTVSS
4C12 VL nucleotide sequence
(SEQ ID NO: 7)
GATATCCAGATGACACAGACTACATCCTCCCTGTCTGCCTCTCTGGGAGA
CAGAGTCACCATCAGTTGCAGGGCAAGTCAGGACATTAGCAATTATTTAA
ACTGGTATCAGCAGAAACCAGATGGAACTGTTAAACTCCTAATCTACTAC
ACATCAAGATTACGCTCAGGAGTCCCATCAAGGTTCAGTGGCAGTGGGTC
TGGAACAGATTATTCTCTCACCATTAACAACCTGGAGCAAGAAGATATTG
CCACTTACTTTTGCCAACAGGGTAATACGCTTCCATTCACGTTCGGCTCG
GGGACAAAGTTGGAAATCAAA
4C12 VL amino acid sequence
(SEQ ID NO: 8)
DIQMTQTTSSLSASLGDRVTISCRASQDISNYLNWYQQKPDGTVKLLIYY
TSRLRSGVPSRFSGSGSGTDYSLTINNLEQEDIATYFCQQGNTLPFTFGS
GTKLEIK

| | CDR1 | CDR2 | CDR3 |
|---|---|---|---|
| | residues | residues | residues |
| 4C12 VH (SEQ ID NO: 6) | 26-33 | 51-58 | 97-109 |
| 4C12 VL (SEQ ID NO: 8) | 27-32 | 50-52 | 89-97 |

Provided herein are monoclonal antibodies that bind (such as specifically bind) EBV gp42. In some embodiments, the gp42-specific monoclonal antibody has a Kd of 60 nM or less, 50 nM or less, 40 nM or less, 30 nM or less, 20 nM or less, 15 nM or less, 14 nM or less, 13 nM or less, 12 nM or less, 11 nM or less, 10 nM or less, 5 nM or less, 1 nM or less or 0.5 nM or less. In some embodiments, the monoclonal antibody includes a variable heavy (VH) domain and a variable light (VL) domain. In some examples, the monoclonal antibody includes at least a portion of the amino acid sequence set forth herein as SEQ ID NO: 2 and/or SEQ ID NO: 4, such as one or more (such as all three) CDR sequences from SEQ ID NO: 2 and/or SEQ ID NO: 4, as determined by any numbering scheme, such as IMGT, Kabat or Chothia, or any combination thereof. In other examples, the monoclonal antibody includes at least a portion of the amino acid sequence set forth herein as SEQ ID NO: 6 and/or SEQ ID NO: 8, such as one or more (such as all three) CDR sequences from SEQ ID NO: 6 and/or SEQ ID NO: 8, as determined by any numbering scheme, such as IMGT, Kabat or Chothia, or any combination thereof. In other examples, the monoclonal antibody includes at least a portion of the amino acid sequence set forth herein as SEQ ID NO: 10 and/or SEQ ID NO: 11, such as one or more (such as all three) CDR sequences from SEQ ID NO: and/or SEQ ID NO: 11, as determined by any numbering scheme, such as IMGT, Kabat or Chothia, or any combination thereof. In other examples, the monoclonal antibody includes at least a portion of the amino acid sequence set forth herein as SEQ ID NO: 12 and/or SEQ ID NO: 13, such as one or more (such as all three) CDR sequences from SEQ ID NO: 12 and/or SEQ ID NO: 13, as determined by any numbering scheme, such as IMGT, Kabat or Chothia, or any combination thereof. In other examples, the monoclonal antibody includes at least a portion of the amino acid sequence set forth herein as SEQ ID NO: 14 and/or SEQ ID NO: 15, such as one or more (such as all three) CDR sequences from SEQ ID NO: 14 and/or SEQ ID NO: 15, as determined by any numbering scheme, such as IMGT, Kabat or Chothia, or any combination thereof. In other examples, the monoclonal antibody includes at least a portion of the amino acid sequence set forth herein as SEQ ID NO: 16 and/or SEQ ID NO: 17, such as one or more (such as all three) CDR sequences from SEQ ID NO: 16 and/or SEQ ID NO: 17, as determined by any numbering scheme, such as IMGT, Kabat or Chothia, or any combination thereof. In other examples, the monoclonal antibody includes at least a portion of the amino acid sequence set forth herein as SEQ ID NO: 18 and/or SEQ ID NO: 19, such as one or more (such as all three) CDR sequences from SEQ ID NO: 18 and/or SEQ ID NO: 19, as determined by any numbering scheme, such as IMGT, Kabat or Chothia, or any combination thereof. In other examples, the monoclonal antibody includes at least a portion of the amino acid sequence set forth herein as SEQ ID NO: 20 and/or SEQ ID NO: 21, such as one or more (such as all three) CDR sequences from SEQ ID NO: 20 and/or SEQ ID NO: 21, as determined by any numbering scheme, such as IMGT, Kabat or Chothia, or any combination thereof. In other examples, the monoclonal antibody includes at least a portion of the amino acid sequence set forth herein as SEQ ID NO: 22 and/or SEQ ID NO: 23, such as one or more (such as all three) CDR sequences from SEQ ID NO: 22 and/or SEQ ID NO: 23, as determined by any numbering scheme, such as IMGT, Kabat or Chothia, or any combination thereof. In yet other examples, the monoclonal antibody includes at least a portion of the amino acid sequence set forth herein as SEQ ID NO: 24 and/or SEQ ID NO: 25, such as one or more (such as all three) CDR sequences from SEQ ID NO: 24 and/or SEQ ID NO: 25, as determined by any numbering scheme, such as IMGT, Kabat or Chothia, or any combination thereof.

In some embodiments, the VH domain of the monoclonal antibody comprises the CDR1, CDR2 and CDR3 sequences of SEQ ID NO: 2, SEQ ID NO: 10, SEQ ID NO: 12, SEQ ID NO: 14, SEQ ID NO: 16, SEQ ID NO: 18, SEQ ID NO: 20, SEQ ID NO: 22 or SEQ ID NO: 24 and/or the VL domain of the monoclonal antibody comprises the CDR1, CDR2 and CDR3 sequences of SEQ ID NO: 4, SEQ ID NO: 11, SEQ ID NO: 13, SEQ ID NO: 15, SEQ ID NO: 17, SEQ ID NO: 19, SEQ ID NO: 21, SEQ ID NO: 23 or SEQ ID NO: 25. In other embodiments, the VH domain of the monoclonal antibody comprises the CDR1, CDR2 and CDR3 sequences of SEQ ID NO: 6 and/or the VL domain of the monoclonal antibody comprises the CDR1, CDR2 and CDR3 sequences of SEQ ID NO: 8. In some examples, the CDR sequences are determined using the IMGT, Kabat or Chothia numbering scheme, or a combination thereof. In particular examples, the CDR sequences are determined using IMGT.

In some embodiments, the CDR1, CDR2 and CDR3 sequences of the VH domain of the monoclonal antibody respectively comprise residues 26-35, 53-61 and 100-109 of SEQ ID NO: 2, SEQ ID NO: 10, SEQ ID NO: 12, SEQ ID NO: 14, SEQ ID NO: 16, SEQ ID NO: 18, SEQ ID NO: 20, SEQ ID NO: 22 or SEQ ID NO: 24; and/or the CDR1, CDR2 and CDR3 sequences of the VL domain of the monoclonal antibody respectively comprise residues 26-31, 49-51 and 88-99 of SEQ ID NO: 4, SEQ ID NO: 11, SEQ ID NO: 13, SEQ ID NO: 15, SEQ ID NO: 17, SEQ ID NO: 19, SEQ ID NO: 21, SEQ ID NO: 23 or SEQ ID NO: 25. In other embodiments, the CDR1, CDR2 and CDR3 sequences of the VH domain of the monoclonal antibody respectively comprise residues 26-33, 51-58 and 97-109 of SEQ ID NO: 6; and/or the CDR1, CDR2 and CDR3 sequences of the VL domain of the monoclonal antibody respectively comprise residues 27-32, 50-52 and 89-97 of SEQ ID NO: 8.

In some examples, the amino acid sequence of the VH domain of the monoclonal antibody is at least 80%, at least 85%, at least 90%, at least 95%, at least 96%, at least 97%, at least 98% or at least 99% identical to SEQ ID NO: 2 and/or the amino acid sequence of the VL domain of the monoclonal antibody is at least 80%, at least 85%, at least 90%, at least 95%, at least 96%, at least 97%, at least 98% or at least 99% identical to SEQ ID NO: 4. In specific non-limiting examples, the sequence of the VH domain of the monoclonal antibody comprises or consists of SEQ ID NO: 2, and/or the sequence of the VL domain of the monoclonal antibody comprises or consists of SEQ ID NO: 4.

In some examples, the amino acid sequence of the VH domain of the monoclonal antibody is at least 80%, at least 85%, at least 90%, at least 95%, at least 96%, at least 97%, at least 98% or at least 99% identical to SEQ ID NO: 10 and/or the amino acid sequence of the VL domain of the monoclonal antibody is at least 80%, at least 85%, at least 90%, at least 95%, at least 96%, at least 97%, at least 98% or at least 99% identical to SEQ ID NO: 11. In specific non-limiting examples, the sequence of the VH domain of the monoclonal antibody comprises or consists of SEQ ID NO: 10, and/or the sequence of the VL domain of the monoclonal antibody comprises or consists of SEQ ID NO: 11.

In some examples, the amino acid sequence of the VH domain of the monoclonal antibody is at least 80%, at least 85%, at least 90%, at least 95%, at least 96%, at least 97%, at least 98% or at least 99% identical to SEQ ID NO: 12 and/or the amino acid sequence of the VL domain of the monoclonal antibody is at least 80%, at least 85%, at least 90%, at least 95%, at least 96%, at least 97%, at least 98% or at least 99% identical to SEQ ID NO: 13. In specific non-limiting examples, the sequence of the VH domain of the monoclonal antibody comprises or consists of SEQ ID NO: 12, and/or the sequence of the VL domain of the monoclonal antibody comprises or consists of SEQ ID NO: 13.

In some examples, the amino acid sequence of the VH domain of the monoclonal antibody is at least 80%, at least 85%, at least 90%, at least 95%, at least 96%, at least 97%, at least 98% or at least 99% identical to SEQ ID NO: 14 and/or the amino acid sequence of the VL domain of the monoclonal antibody is at least 80%, at least 85%, at least 90%, at least 95%, at least 96%, at least 97%, at least 98% or at least 99% identical to SEQ ID NO: 15. In specific non-limiting examples, the sequence of the VH domain of the monoclonal antibody comprises or consists of SEQ ID NO: 14, and/or the sequence of the VL domain of the monoclonal antibody comprises or consists of SEQ ID NO: 15.

In some examples, the amino acid sequence of the VH domain of the monoclonal antibody is at least 80%, at least 85%, at least 90%, at least 95%, at least 96%, at least 97%, at least 98% or at least 99% identical to SEQ ID NO: 16 and/or the amino acid sequence of the VL domain of the monoclonal antibody is at least 80%, at least 85%, at least 90%, at least 95%, at least 96%, at least 97%, at least 98% or at least 99% identical to SEQ ID NO: 17. In specific non-limiting examples, the sequence of the VH domain of the monoclonal antibody comprises or consists of SEQ ID NO: 16, and/or the sequence of the VL domain of the monoclonal antibody comprises or consists of SEQ ID NO: 7.

In some examples, the amino acid sequence of the VH domain of the monoclonal antibody is at least 80%, at least 85%, at least 90%, at least 95%, at least 96%, at least 97%, at least 98% or at least 99% identical to SEQ ID NO: 18 and/or the amino acid sequence of the VL domain of the monoclonal antibody is at least 80%, at least 85%, at least 90%, at least 95%, at least 96%, at least 97%, at least 98% or at least 99% identical to SEQ ID NO: 19. In specific non-limiting examples, the sequence of the VH domain of the monoclonal antibody comprises or consists of SEQ ID NO: 18, and/or the sequence of the VL domain of the monoclonal antibody comprises or consists of SEQ ID NO: 19.

In some examples, the amino acid sequence of the VH domain of the monoclonal antibody is at least 80%, at least 85%, at least 90%, at least 95%, at least 96%, at least 97%, at least 98% or at least 99% identical to SEQ ID NO: 20 and/or the amino acid sequence of the VL domain of the monoclonal antibody is at least 80%, at least 85%, at least 90%, at least 95%, at least 96%, at least 97%, at least 98% or at least 99% identical to SEQ ID NO: 21. In specific non-limiting examples, the sequence of the VH domain of the monoclonal antibody comprises or consists of SEQ ID NO: 20, and/or the sequence of the VL domain of the monoclonal antibody comprises or consists of SEQ ID NO: 21.

In some examples, the amino acid sequence of the VH domain of the monoclonal antibody is at least 80%, at least 85%, at least 90%, at least 95%, at least 96%, at least 97%, at least 98% or at least 99% identical to SEQ ID NO: 22 and/or the amino acid sequence of the VL domain of the monoclonal antibody is at least 80%, at least 85%, at least 90%, at least 95%, at least 96%, at least 97%, at least 98% or at least 99% identical to SEQ ID NO: 23. In specific non-limiting examples, the sequence of the VH domain of the monoclonal antibody comprises or consists of SEQ ID NO: 22, and/or the sequence of the VL domain of the monoclonal antibody comprises or consists of SEQ ID NO: 23.

In some examples, the amino acid sequence of the VH domain of the monoclonal antibody is at least 80%, at least 85%, at least 90%, at least 95%, at least 96%, at least 97%, at least 98% or at least 99% identical to SEQ ID NO: 24 and/or the amino acid sequence of the VL domain of the monoclonal antibody is at least 80%, at least 85%, at least 90%, at least 95%, at least 96%, at least 97%, at least 98% or at least 99% identical to SEQ ID NO: 25. In specific non-limiting examples, the sequence of the VH domain of the monoclonal antibody comprises or consists of SEQ ID NO: 24, and/or the sequence of the VL domain of the monoclonal antibody comprises or consists of SEQ ID NO: 25.

In other examples, the amino acid sequence of the VH domain of the monoclonal antibody is at least 80%, at least 85%, at least 90%, at least 95%, at least 96%, at least 97%, at least 98% or at least 99% identical to SEQ ID NO: 6 and/or the amino acid sequence of the VL domain of the monoclonal antibody is at least 80%, at least 85%, at least 90%, at least 95%, at least 96%, at least 97%, at least 98% or at least 99% identical to SEQ ID NO: 8. In specific non-limiting examples, the sequence of the VH domain of the monoclonal antibody comprises or consists of SEQ ID NO: 6, and/or the sequence of the VL domain of the monoclonal antibody comprises or consists of SEQ ID NO: 8.

In some embodiments, the monoclonal antibody is an antigen-binding fragment, such as an Fab fragment, an Fab′ fragment, an F(ab)′₂ fragment, a single chain variable fragment (scFv), or a disulfide stabilized variable fragment (dsFv).

In some embodiments, the monoclonal antibody is an IgG, such as IgG1.

In some embodiments, the monoclonal antibody is a mouse antibody. In other embodiments, the monoclonal antibody is a non-human primate antibody. In other embodiments, the monoclonal antibody is a humanized antibody. In yet other embodiments, the monoclonal antibody is a chimeric antibody.

In some embodiments, the monoclonal antibody further includes a constant region, such as an IgG constant region, for example, an IgG1 constant region, such as a human IgG1 constant region. In some examples, the constant region includes at least one amino acid modification to increase the half-life, stability and/or function of the monoclonal antibody. Section V below provides an exemplary list constant region modifications.

Also provided herein are conjugates that include a gp42-specific monoclonal antibody disclosed herein linked to an effector molecule or a detectable label. In some embodiments, the detectable marker is a fluorescent, enzymatic, radioactive or nucleic acid label.

Further provided herein are bispecific antibodies. In some embodiments, the bispecific antibody comprising a gp42-specific monoclonal antibody disclosed herein (such as A10 or 4C12, or humanized versions thereof) and a second monoclonal antibody or antigen-binding fragment thereof. In some examples, the second monoclonal antibody binds a different epitope of gp42 (such as an A10-4C12 bispecific antibody, or a humanized A10-4C12 bispecific antibody). In other examples, the second monoclonal antibody binds an EBV protein other than gp42, such as gp350, gH/gL, gH/gL/gp42 or gB.

Also provided herein are fusion proteins that include a gp42-specific monoclonal antibody disclosed herein and a heterologous protein. In some examples, the heterologous protein is an Fc protein, such as human Fc. In other examples, the heterologous protein is a protein tag, such as a myc tag, His tag, HA tag, or FLAG tag. In other examples, the heterologous protein is an affinity tag, such as chitin binding protein, maltose binding protein, or glutathione-S-transferase (GST).

Further provided herein are nucleic acid molecules that encode a gp42-specific monoclonal antibody disclosed herein, or a domain thereof (such as a VH domain or a VL domain). In some examples, the nucleic acid molecule includes the nucleotide sequence of SEQ ID NO: 1, or a degenerate variant thereof; the nucleotide sequence of SEQ ID NO: 3, or a degenerate variant thereof; or the nucleotide sequences of SEQ ID NO: 1 and SEQ ID NO: 3, or degenerate variants thereof. In other examples, the nucleic acid molecule includes the nucleotide sequence of SEQ ID NO: 5, or a degenerate variant thereof; the nucleotide sequence of SEQ ID NO: 7, or a degenerate variant thereof; or the nucleotide sequences of SEQ ID NO: 5 and SEQ ID NO: 7, or degenerate variants thereof. In some embodiments, the nucleic acid molecule is operably linked to a promoter, such as a heterologous promoter. Also provided are host cells, such as a mammalian cell, insect cell, bacterial cell, or chicken cell, which includes such nucleic acid molecules. In some embodiments, the nucleic acid molecule encodes a VH domain comprising or consisting of the amino acid sequence of any one of SEQ ID NOs: 2, 6, 10, 12, 14, 16, 18, 20, 22 and 24 and/or a VL domain comprising or consisting of the amino acid sequence of any one of SEQ ID NOs: 4, 8, 11, 13, 15, 17, 19, 21, 23 and 25.

Also provided are vectors, such as a plasmid or viral vector (such as AAV or a lentiviral vector), that include a nucleic acid molecule disclosed herein, such as one encoding a gp42-specific monoclonal antibody disclosed herein. Also provided are host cells, such as a mammalian cell, insect cell, bacterial cell, or chicken cell, which includes such vectors.

Further provided herein are compositions that include a monoclonal antibody, conjugate, bispecific antibody, fusion protein, cell, nucleic acid molecule or vector disclosed herein, and a pharmaceutically acceptable carrier.

Methods of detecting EBV in a subject are also provided herein. In some embodiments, the method includes contacting a biological sample from the subject with a gp42-specific monoclonal antibody disclosed herein under conditions sufficient to form an immune complex; and detecting the presence of the immune complex in the sample (for example using a labeled secondary antibody that can bind to a gp42-specific monoclonal antibody disclosed herein). The presence of the immune complex in the sample detects EBV in the subject. In some examples, the biological sample is a fluid sample, such as but not limited to blood, plasma, serum, urine, saliva and cerebrospinal fluid. In other examples, the biological sample is a tissue or cell sample, such as a sample comprising B cells or lymphoid tissue.

Methods of diagnosing a subject as having an EBV infection are also provided herein. In some embodiments, the method includes contacting a biological sample from the subject with a gp42-specific monoclonal antibody disclosed herein under conditions sufficient to form an immune complex; and detecting the presence of the immune complex in the sample (for example using a labeled secondary antibody that can bind to a gp42-specific monoclonal antibody disclosed herein). The presence of the immune complex in the sample the subject diagnoses the subject as having an EBV infection. In some examples, the biological sample is a fluid sample, such as but not limited to blood, plasma, serum, urine, saliva and cerebrospinal fluid. In other examples, the biological sample is a tissue or cell sample, such as a sample comprising B cells or lymphoid tissue.

Also provided are methods for the treatment or prophylaxis of an EBV infection in a subject. In some embodiments, the method includes administering to the subject a therapeutically effective amount of a monoclonal antibody, conjugate, bispecific antibody, fusion protein, nucleic acid molecule, vector, or a composition disclosed herein. In some examples, the method further includes administering to the subject one or more additional antibodies that specifically bind to an EBV protein, or one or more nucleic acid molecules encoding the additional antibodies. In particular examples, the EBV protein to which the additional antibody binds is gp42, gp350, gH/gL, gH/gL/gp42, or gB. In some examples, the method is a prophylactic method and the subject is seronegative for EBV. In some examples, the method is a prophylactic method and the subject is seropositive for EBV. In some examples, the method is a prophylactic method and the subject is immunosuppressed. In some examples, the method is a prophylactic method and the subject is immunocompromised. In some examples, the method is a therapeutic method and the subject is seronegative for EBV. In some examples, the method is a therapeutic method and the subject is seropositive for EBV. In some examples, the method is a therapeutic method and the subject is immunosuppressed. In some examples, the method is a therapeutic method and the subject is immunocompromised. In specific non-limiting examples, the subject has been diagnosed with an EBV infection. In particular examples, the subject has received an organ or tissue transplant.

Further provided are methods of preventing or inhibiting EBV-associated lymphoproliferative disease in a subject. In some embodiments, the method includes administering to the subject a therapeutically effective amount of a monoclonal antibody, conjugate, bispecific antibody, fusion protein, nucleic acid molecule, vector, or a composition disclosed herein. In some examples, the method further includes administering to the subject one or more additional antibodies that specifically bind to an EBV protein, or one or more nucleic acid molecules encoding the additional antibodies. In particular examples, the EBV protein to which the additional antibody binds is gp42, gp350, gH/gL, gH/gL/gp42, or gB. In some examples, the method is a prophylactic method and the subject is seronegative for EBV. In some examples, the method is a prophylactic method and the subject is seropositive for EBV. In some examples, the method is a prophylactic method and the subject is immunosuppressed. In some examples, the method is a prophylactic method and the subject is immunocompromised. In some examples, the method is a therapeutic method and the subject is seronegative for EBV. In some examples, the method is a therapeutic method and the subject is seropositive for EBV. In some examples, the method is a therapeutic method and the subject is immunosuppressed. In some examples, the method is a therapeutic method and the subject is immunocompromised. In specific non-limiting examples, the subject has been diagnosed with an EBV infection. In particular examples, the subject has received an organ or tissue transplant.

Further provided herein are kits that include a monoclonal antibody, conjugate, bispecific antibody, fusion protein, nucleic acid molecule, or vector disclosed herein; and buffer, one or more detection reagents and/or instructional materials.

### IV. Monoclonal Antibodies and Antigen-Binding Fragments Thereof

The monoclonal antibodies disclosed herein can include any suitable framework region, such as (but not limited to) a human framework region. Human framework regions, and mutations that can be made in human antibody framework regions, have been described (see, for example, U.S. Pat. No. 5,585,089, which is incorporated herein by reference). Alternatively, a heterologous framework region, such as, but not limited to a mouse or non-human primate framework region, can be included in the heavy or light chain of the antibodies (see, for example, Jones et al., Nature 321:522, 1986; Riechmann et al., Nature 332:323, 1988; Verhoeyen et al., Science 239:1534, 1988; Carter et al., Proc. Natl. Acad. Sci. U.S.A. 89:4285, 1992; Sandhu, Crit. Rev. Biotech. 12:437, 1992; and Singer et al., J. Immunol. 150:2844, 1993.)

The monoclonal antibodies disclosed herein can be of any isotype. The monoclonal antibody can be, for example, an IgM or an IgG antibody, such as IgG₁, IgG₂, IgG₃, or IgG₄. The class of an antibody that specifically binds gp42 can be switched with another (for example, IgG can be switched to IgM). Class switching can also be used to convert one IgG subclass to another, such as from IgG₁ to IgG2. In one aspect, a nucleic acid molecule encoding a VL or a VH is isolated such that it does not include any nucleic acid sequences encoding the constant region of the light or heavy chain, respectively. A nucleic acid molecule encoding V_{L} or V_{H} is then operatively linked to a nucleic acid sequence encoding a C_{L} or C_{H} from a different class of immunoglobulin molecule. This can be achieved using a vector or nucleic acid molecule that comprises a C_{L} or C_{H} chain.

In some examples, the disclosed antibodies are oligomers of antibodies, such as dimers, trimers, tetramers, pentamers, hexamers, septamers, octomers and so on.

Antibody fragments are also encompassed by the present disclosure, such as single-domain antibodies (e.g., VH domain antibodies), Fab, F(ab′)₂, Fv and scFv. These antibody fragments retain the ability to selectively bind with the antigen. These antigen-binding fragments include:
(1) Fab, the fragment which contains a monovalent antigen-binding fragment of an antibody molecule, can be produced by digestion of whole antibody with the enzyme papain to yield an intact light chain and a portion of one heavy chain;
(2) Fab′, the fragment of an antibody molecule can be obtained by treating whole antibody with pepsin, followed by reduction, to yield an intact light chain and a portion of the heavy chain; two Fab′ fragments are obtained per antibody molecule;
(3) (Fab′)₂, the fragment of the antibody that can be obtained by treating whole antibody with the enzyme pepsin without subsequent reduction; F(ab′)₂ is a dimer of two Fab′ fragments held together by two disulfide bonds;
(4) Fv, a genetically engineered fragment containing the variable region of the light chain and the variable region of the heavy chain expressed as two chains;
(5) Single chain antibody (such as scFv), a genetically engineered molecule containing the variable region of the light chain, the variable region of the heavy chain, linked by a suitable polypeptide linker as a genetically fused single chain molecule;
(6) A dimer of a single chain antibody (scFV₂), defined as a dimer of a scFv (also known as a “miniantibody”); and
(7) VH single-domain antibody, an antibody fragment consisting of the heavy chain variable domain.

Methods of making these fragments have been described (see for example, Harlow and Lane, Antibodies: A Laboratory Manual, Cold Spring Harbor Laboratory, New York, 1988).

In some cases, antibody fragments can be prepared by proteolytic hydrolysis of the antibody or by expression in a host cell (such as E. coli) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly (see U.S. Pat. Nos. 4,036,945 and 4,331,647).

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

### V. Antibody Variants

The gp42-specific monoclonal antibodies described herein can include one or more amino acid sequence changes, such as one more amino acid substitutions, deletions or insertions (such as at least 2, at least 3, at least 4, at least 5 or at least 10 amino acid substitutions, such as 1-20, 1-15, 1-10, 2-20, 2-10, 1-5, or 5-10 amino acid substitutions, such as 1, 2, 3, 4, 5, 6, 7, 8, 9, 10, 11, 12, 13, 14, 15, 16, 17, 18, 19 or 20 amino acid substitutions). For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody (such as at least 2, at least 3, at least 4, at least 5 or at least 10 amino acid insertions, substitutions, and/or deletions, such as 1-20, 1-15, 1-10, 2-20, 2-10, 1-5, or 5-10 amino acid insertions, substitutions, and/or deletions, such as 1, 2, 3, 4, 5, 6, 7, 8, 9, 10, 11, 12, 13, 14, 15, 16, 17, 18, 19 or 20 amino acid insertions, substitutions, and/or deletions). Any combination of deletion, insertion, and substitution can be made to arrive at the final construct, provided that the final construct possesses the desired characteristics, for example, antigen-binding.

Conservative variants of the disclosed antibodies can be produced. Such conservative variants will retain critical amino acid residues necessary for correct folding and stabilization between the V_{H} and the V_{L} regions, and will retain the charge characteristics of the residues in order to preserve the low pI and low toxicity of the molecules Amino acid substitutions (such as at most one, at most two, at most three, at most four, or at most five amino acid substitutions) can be made in the V_{H} and/or the V_{L} regions to increase yield. Conservative amino acid substitution tables providing functionally similar amino acids can be used to select an appropriate substitution. The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

In certain embodiments, antibody variants having one or more amino acid substitutions are provided. Sites of interest for substitutional mutagenesis include the CDRs and the framework regions Amino acid substitutions may be introduced into an antibody of interest and the products screened for a desired activity, for example, retained/improved antigen (gp42) binding, decreased immunogenicity, improved effector functions (such as ADCC or CDC), or increased potency.

In some embodiments, the VH domain of the antibody includes up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as SEQ ID NO: 2, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24 or SEQ ID NO: 6. In some embodiments, the VL domain of the antibody includes up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as SEQ ID NO: 4, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25 or SEQ ID NO: 8.

In some embodiments, the antibody or antigen binding fragment can include up to 10 (such as up to 1, up to 2, up to 3, up to 4, up to 5, up to 6, up to 7, up to 8, or up to 9) amino acid substitutions (such as conservative amino acid substitutions) in the framework regions of the VH domain of the antibody, or the VL domain of the antibody, or the VH and VL domains of the antibody, compared to any one of SEQ ID NOs: 2, 4, 6, 8 and 10-25.

In certain embodiments, substitutions, insertions, or deletions may occur within one or more CDRs so long as such alterations do not substantially reduce the ability of the antibody to bind gp42. For example, conservative alterations (such as conservative substitutions as provided herein) that do not substantially reduce binding affinity may be made in CDRs. In certain embodiments of the variant V_{H} and V_{L} sequences described herein, each CDR either is unaltered, or contains no more than one, two or three amino acid substitutions.

To increase binding affinity of the antibody, the VL and VH segments can be randomly mutated, such as within HCDR3 region or the LCDR3 region, in a process analogous to the in vivo somatic mutation process responsible for affinity maturation of antibodies during a natural immune response. Thus in vitro affinity maturation can be accomplished by amplifying VH and VL regions using PCR primers complementary to the HCDR3 or LCDR3, respectively. In this process, the primers have been “spiked” with a random mixture of the four nucleotide bases at certain positions such that the resultant PCR products encode V_{H} and V_{L} segments into which random mutations have been introduced into the V_{H} and/or V_{L} CDR3 regions. These randomly mutated VH and VL segments can be tested to determine the binding affinity for EBV gp42. Methods of in vitro affinity maturation have been described (see, for example, Chowdhury, Methods Mol. Biol. 207:179-196, 2008; and Hoogenboom et al., in Methods in Molecular Biology 178:1-37 (O'Brien et al., ed., Human Press, Totowa, N.J., 2001).

In certain embodiments, an antibody or antigen binding fragment is altered to increase or decrease the extent to which the antibody or antigen binding fragment is glycosylated. Addition or deletion of glycosylation sites may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region (see, for example, Wright et al., TIBTECH 15:26-32, 1997). The oligosaccharide may include various carbohydrates, for example, mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody may be made in order to create antibody variants with certain improved properties.

In one embodiment, antibody variants are provided having a carbohydrate structure that lacks fucose attached (directly or indirectly) to an Fc region. For example, the amount of fucose in such an antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (for example, complex, hybrid and high mannose structures) as measured by MALDI-TOF mass spectrometry, as described in WO 2008/077546, for example. Asn297 refers to the asparagine residue located at about position 297 in the Fc region; however, Asn297 may also be located about ±3 amino acids upstream or downstream of position 297, such as between positions 294 and 300, due to minor sequence variations in antibodies. Such fucosylation variants may have improved ADCC function (see, for example, US Patent Publication Nos. US 2003/0157108 and US 2004/0093621). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); and Yamane-Ohnuki et al. Biotech. Bioeng. 87: 614, 2004. Examples of cell lines capable of producing defucosylated antibodies include Lec 13 CHO cells deficient in protein fucosylation (Ripka et al. Arch. Biochem. Biophys. 249:533-545 (1986); US Patent Application No. US 2003/0157108; and WO 2004/056312, especially at Example 11), and knockout cell lines, such as alpha-1,6-fucosyltransferase gene, FUT8, knockout CHO cells (see, for example, Yamane-Ohnuki et al. Biotech. Bioeng. 87: 614, 2004; Kanda et al., Biotechnol. Bioeng., 94(4):680-688 (2006); and WO2003/085107).

Antibody variants with bisected oligosaccharides are further provided, for example, in which a biantennary oligosaccharide attached to the Fc region of the antibody is bisected by GlcNAc. Such antibody variants may have reduced fucosylation and/or improved ADCC function. Examples of such antibody variants are described, for example, in WO 2003/011878; U.S. Pat. No. 6,602,684; and US 2005/0123546. Antibody variants with at least one galactose residue in the oligosaccharide attached to the Fc region are also provided. Such antibody variants may have improved CDC function. Such antibody variants are described, for example, in WO 1997/30087; WO 1998/58964; and WO 1999/22764.

In several embodiments, the constant region of the antibody includes one or more amino acid substitutions to optimize in vivo half-life of the antibody. The serum half-life of IgG antibodies is regulated by the neonatal Fc receptor (FcRn). Thus, in several embodiments, the antibody includes an amino acid substitution that increases binding to the FcRn. Exemplary substitutions that enhance binding to FcRn are known, and can be made to the gp42 antibodies provided herein, and include substitutions at IgG constant regions T250Q and M428L (see, for example, Hinton et al., J Immunol., 176:346-356, 2006); M428L and N434S (the “LS” mutation, see, for example, Zalevsky, et al., Nature Biotechnology, 28:157-159, 2010); N434A (see, for example, Petkova et al., Int. Immunol., 18:1759-1769, 2006); T307A, E380A, and N434A (see, for example, Petkova et al., Int. Immunol., 18:1759-1769, 2006); and M252Y, S254T, and T256E (see, for example, Dall'Acqua et al., J. Biol. Chem., 281:23514-23524, 2006). The disclosed antibodies and antigen binding fragments can be linked to an Fc polypeptide including any of the substitutions listed above, for example, the Fc polypeptide can include the M428L and N434S substitutions.

In some embodiments, the constant region of the antibody includes one of more amino acid substitutions to optimize antibody-dependent cell-mediated cytotoxicity (ADCC). ADCC is mediated primarily through a set of closely related Fcγ receptors. In some embodiments, the antibody includes one or more amino acid substitutions that increase binding to FcγRIIIa. Several such substitutions are known, for example substitutions at IgG constant regions S239D and I332E (see, for example, Lazar et al., Proc. Natl., Acad. Sci. U.S.A., 103:4005-4010, 2006); and S239D, A330L, and I332E (see, for example, Lazar et al., Proc. Natl., Acad. Sci. U.S.A., 103:4005-4010, 2006).

Combinations of the above substitutions are also included, to generate an IgG constant region with increased binding to FcRn and FcγRIIIa. The combinations increase antibody half-life and ADCC. For example, such combination include antibodies with the following amino acid substitutions in the Fc region:
(1) S239D/I332E and T250Q/M428L;
(2) S239D/I332E and M428L/N434S;
(3) S239D/I332E and N434A;
(4) S239D/I332E and T307A/E380A/N434A;
(5) S239D/I332E and M252Y/S254T/T256E;
(6) S239D/A330L/I332E and T250Q/M428L;
(7) S239D/A330L/I332E and M428L/N434S;
(8) S239D/A330L/I332E and N434A;
(9) S239D/A330L/I332E and T307A/E380A/N434A; or
(10) S239D/A330L/I332E and M252Y/S254T/T256E.

In some examples, the antibodies, or an antigen binding fragment thereof, is modified such that it is directly cytotoxic to infected cells, or uses natural defenses such as complement, ADCC, or phagocytosis by macrophages.

In certain embodiments, an antibody provided herein may be further modified to contain additional nonproteinaceous moieties. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (such as glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

The antibody or antigen binding fragment can be derivatized or linked to another molecule (such as another peptide or protein). In general, the antibody or antigen binding fragment is derivatized such that the binding to EBV gp42 is not affected adversely by the derivatization or labeling. For example, the antibody or antigen binding fragment can be functionally linked (by chemical coupling, genetic fusion, noncovalent association or otherwise) to one or more other molecular entities, such as another antibody (for example, a bi-specific antibody or a diabody), a detectable marker, an effector molecule, or a protein or peptide that can mediate association of the antibody or antibody portion with another molecule (such as a streptavidin core region or a polyhistidine tag).

### VI. Antibody Conjugates

The gp42-specific monoclonal antibodies disclosed herein can be conjugated to an agent, such as an effector molecule or detectable label, using any number of suitable means. Both covalent and noncovalent attachment means may be used. Various effector molecules and detectable markers can be used, including (but not limited to) toxins and radioactive agents such as ¹²⁵I, ³²P, ¹⁴C, ³H and ³⁵S and other labels, targeting moieties and ligands.

The choice of a particular effector molecule or detectable marker depends on the particular target molecule or cell, and the desired biological effect. Thus, for example, the effector molecule can be a cytotoxin that is used to bring about the death of a particular target cell (such as an EBV infected cell). In other embodiments, the effector molecule can be a cytokine, such as IL-15; conjugates including the cytokine can be used, for example, to stimulate immune cells locally.

The procedure for attaching an effector molecule or detectable marker to an antibody or antigen binding fragment varies according to the chemical structure of the effector. Polypeptides typically contain a variety of functional groups; such as carboxylic acid (COOH), free amine (—NH₂) or sulfhydryl (—SH) groups, which are available for reaction with a suitable functional group on a polypeptide to result in the binding of the effector molecule or detectable marker. Alternatively, the antibody or antigen binding fragment is derivatized to expose or attach additional reactive functional groups. The derivatization may involve attachment of any of a number of known linker molecules such as those available from Pierce Chemical Company, Rockford, IL. The linker can be any molecule used to join the antibody or antigen binding fragment to the effector molecule or detectable marker. The linker is capable of forming covalent bonds to both the antibody/antigen binding fragment and to the effector molecule/detectable marker. Suitable linkers include, but are not limited to, straight or branched-chain carbon linkers, heterocyclic carbon linkers, or peptide linkers. Where the antibody or antigen binding fragment and the effector molecule or detectable marker are polypeptides, the linkers may be joined to the constituent amino acids through their side groups (such as through a disulfide linkage to cysteine) or to the alpha carbon amino and carboxyl groups of the terminal amino acids.

The antibody or antigen binding fragment can be conjugated with a detectable marker; for example, a detectable marker capable of detection by ELISA, spectrophotometry, flow cytometry, microscopy or diagnostic imaging techniques (such as computed tomography (CT), computed axial tomography (CAT) scans, magnetic resonance imaging (MRI), nuclear magnetic resonance imaging NMRI), magnetic resonance tomography (MTR), ultrasound, fiberoptic examination, and laparoscopic examination). Specific, non-limiting examples of detectable markers include fluorophores, chemiluminescent agents, enzymatic linkages, radioactive isotopes and heavy metals or compounds (for example super paramagnetic iron oxide nanocrystals for detection by MRI). For example, useful detectable markers include fluorescent compounds, including fluorescein, fluorescein isothiocyanate, rhodamine, 5-dimethylamine−1-napthalenesulfonyl chloride, phycoerythrin, lanthanide phosphors and the like. Bioluminescent markers are also of use, such as luciferase, green fluorescent protein (GFP), and yellow fluorescent protein (YFP). An antibody or antigen binding fragment can also be conjugated with enzymes that are useful for detection, such as horseradish peroxidase, β-galactosidase, luciferase, alkaline phosphatase, glucose oxidase and the like. When an antibody or antigen binding fragment is conjugated with a detectable enzyme, it can be detected by adding additional reagents that the enzyme uses to produce a reaction product that can be discerned. For example, when the agent horseradish peroxidase is present the addition of hydrogen peroxide and diaminobenzidine leads to a colored reaction product, which is visually detectable. An antibody or antigen binding fragment may also be conjugated with biotin, and detected through indirect measurement of avidin or streptavidin binding. It should be noted that the avidin itself can be conjugated with an enzyme or a fluorescent label.

The antibody or antigen binding fragment can be conjugated with a paramagnetic agent, such as gadolinium. Paramagnetic agents such as superparamagnetic iron oxide are also of use as labels. Antibodies can also be conjugated with lanthanides (such as europium and dysprosium), and manganese. An antibody or antigen binding fragment may also be labeled with a predetermined polypeptide epitopes recognized by a secondary reporter (such as leucine zipper pair sequences, binding sites for secondary antibodies, metal binding domains, epitope tags).

The antibody or antigen binding fragment can also be conjugated with a radiolabeled amino acid. The radiolabel may be used for both diagnostic and therapeutic purposes. For instance, the radiolabel may be used to detect EBV gp42 and EBV gp42-expressing cells by x-ray, emission spectra, or other diagnostic techniques. Examples of labels for polypeptides include, but are not limited to, the following radioisotopes or radionucleotides: ³H, ¹⁴C, ¹⁵N, ³⁵S, ⁹⁰Y, ⁹⁹Tc, ¹¹¹In, ¹²⁵I, ¹³¹I.

Any known means of detecting detectable markers can be used. Thus, for example, radiolabels may be detected using photographic film or scintillation counters, fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

The average number of effector molecule or detectable marker moieties per antibody or antigen binding fragment in a conjugate can range, for example, from 1 to 20 moieties per antibody or antigen binding fragment. In certain embodiments, the average number of effector molecule or detectable marker moieties per antibody or antigen binding fragment in a conjugate range from about 1 to about 2, from about 1 to about 3, about 1 to about 8; from about 2 to about 6; from about 3 to about 5; or from about 3 to about 4. The loading (for example, effector molecule/antibody ratio) of an conjugate may be controlled in different ways, for example, by: (i) limiting the molar excess of effector molecule-linker intermediate or linker reagent relative to antibody, (ii) limiting the conjugation reaction time or temperature, (iii) partial or limiting reductive conditions for cysteine thiol modification, (iv) engineering by recombinant techniques the amino acid sequence of the antibody such that the number and position of cysteine residues is modified for control of the number or position of linker-effector molecule attachments.

### VII. Multi-Specific Antibodies

Multi-specific antibodies are recombinant proteins comprised of two or more monoclonal antibodies or antigen-binding fragments of two or more different monoclonal antibodies. For example, bispecific antibodies are comprised of antigen-binding fragments of two different monoclonal antibodies. Thus, bispecific antibodies bind two different antigens (or two different epitopes of the same antigen) and trispecific antibodies bind three different antigens or epitopes. The gp42-specific monoclonal antibodies disclosed herein can be used to generate multi-specific (such as bispecific or trispecific) antibodies that target both gp42 and (for example) another EBV protein, such as gp350, gH/gL, gH/gL/gp42, or gB. In one example, the gp42-specific monoclonal antibodies disclosed herein are used to generate multi-specific (such as bispecific or trispecific) antibodies that target both gp42 and gH/gL, thereby providing a means to treat EBV infection.

Provided herein are multi-specific, such as trispecific or bispecific, monoclonal antibodies comprising a gp42-specific monoclonal antibody. In some embodiments, the multi-specific monoclonal antibody further comprises a monoclonal antibody that specifically binds another protein from EBV. In specific examples, the other EBV protein is gH/gL. In other embodiments, the multi-specific monoclonal antibody further includes a second monoclonal antibody that specifically binds gp42. Also provided are isolated nucleic acid molecules and vectors encoding the multi-specific antibodies, and host cells comprising the nucleic acid molecules or vectors. Multi-specific antibodies comprising a gp42-specific antibody can be used for the treatment or prophylaxis of EBV infection. Thus, provided herein are methods for the treatment or prophylaxis of an EBV infection in a subject by administering to the subject a therapeutically effective amount of the gp42-targeting multi-specific (such as bispecific) antibody. In some examples, the method is a prophylactic method and the subject is seronegative for EBV, seropositive for EBV, immunocompromised or immunosuppressed. In specific non-limiting examples, the subject has received an organ or tissue transplant. In other examples, the method is for treatment of an EBV infection and the subject has been diagnosed with an EBV infection.

### VIII. Polynucleotides and Expression

Nucleic acid molecules (for example, cDNA molecules) encoding the amino acid sequences of antibodies, antigen binding fragments, and conjugates that specifically bind EBV gp42 are provided. Nucleic acids encoding these molecules can readily be produced using the amino acid sequences provided herein (such as the CDR sequences and VH and VL sequences), sequences available in the art (such as framework or constant region sequences), and the genetic code. In several embodiments, a nucleic acid molecule can encode the VH, the VL, or both the VH and VL (for example in a bicistronic expression vector) of a disclosed antibody. In several embodiments, the nucleic acid molecules can be expressed in a host cell (such as a mammalian cell) to produce a disclosed antibody.

The genetic code can be used to construct a variety of functionally equivalent nucleic acids, such as nucleic acids that differ in sequence but which encode the same antibody sequence, or encode a conjugate or fusion protein including the VL and/or VH nucleic acid sequence.

In a non-limiting example, an isolated nucleic acid molecule encodes the VH of a disclosed antibody and includes the nucleic acid sequence set forth as SEQ ID NO: 1 or a degenerate variant thereof, or SEQ ID NO: 5 or degenerate variant thereof. In a non-limiting example, an isolated nucleic acid molecule encodes the VL of a disclosed antibody and includes the nucleic acid sequence set forth as any one of SEQ ID NO: 3 or a degenerate variant thereof, or SEQ ID NO: 7 or a degenerate variant thereof. In a non-limiting example, an isolated nucleic acid molecule encodes the VH and VL of a disclosed antibody and includes the nucleic acid sequences set forth as SEQ ID NOs: 1 and 3; or SEQ ID NOs: 5 and 7. In other non-limiting examples, the nucleic acid molecule encodes a VH domain comprising or consisting of the amino acid sequence of any one of SEQ ID NOs: 2, 6, 10, 12, 14, 16, 18, 20, 22 and 24 and/or a VL domain comprising or consisting of the amino acid sequence of any one of SEQ ID NOs: 4, 8, 11, 13, 15, 17, 19, 21, 23 and 25.

Nucleic acid sequences encoding antibodies, bispecific antibodies, fusion proteins and conjugates that specifically bind EBV gp42 can be prepared by any suitable method including, for example, cloning of appropriate sequences or by direct chemical synthesis by methods such as the phosphotriester method of Narang et al., Meth. Enzymol. 68:90-99, 1979; the phosphodiester method of Brown et al., Meth. Enzymol. 68:109-151, 1979; the diethylphosphoramidite method of Beaucage et al., Tetra. Lett. 22:1859-1862, 1981; the solid phase phosphoramidite triester method described by Beaucage & Caruthers, Tetra. Letts. 22(20):1859-1862, 1981, for example, using an automated synthesizer as described in, for example, Needham-VanDevanter et al., Nucl. Acids Res. 12:6159-6168, 1984; and, the solid support method of U.S. Pat. No. 4,458,066. Chemical synthesis produces a single stranded oligonucleotide. This can be converted into double stranded DNA by hybridization with a complementary sequence or by polymerization with a DNA polymerase using the single strand as a template.

Exemplary nucleic acids can be prepared by cloning techniques. Examples of appropriate cloning and sequencing techniques, and instructions sufficient to direct persons of skill through many cloning exercises are known (see, for example, Sambrook et al. (Molecular Cloning: A Laboratory Manual, 4^{th} ed, Cold Spring Harbor, New York, 2012) and Ausubel et al. (In Current Protocols in Molecular Biology, John Wiley & Sons, New York, through supplement 104, 2013). Product information from manufacturers of biological reagents and experimental equipment also provide useful information. Such manufacturers include the SIGMA Chemical Company (Saint Louis, MO), R&D Systems (Minneapolis, MN), GE Healthcare, Takara Bio USA (Mountain View, CA), Chem Genes Corp., Aldrich Chemical Company (Milwaukee, WI), Glen Research, Inc., Thermo Fisher Scientific, Fisher Scientific, Invitrogen (Carlsbad, CA), and Applied Biosystems (Foster City, CA), as well as many other commercial sources.

Nucleic acids can also be prepared by amplification methods Amplification methods include polymerase chain reaction (PCR), the ligase chain reaction (LCR), the transcription-based amplification system (TAS), the self-sustained sequence replication system (3SR). A wide variety of cloning methods, host cells, and in vitro amplification methodologies can be used.

The nucleic acid molecules can be expressed in a recombinantly engineered host cell such as bacteria, plant, yeast, chicken, insect and mammalian cells. The antibodies and conjugates can be expressed as individual V_{H} and/or V_{L} chain (linked to an effector molecule or detectable marker as needed), or can be expressed as a fusion protein. Methods of expressing and purifying antibodies are known and further described herein (see, for example, Al-Rubeai (ed), Antibody Expression and Production, Springer Press, 2011).

To create a scFv, the V_{H}- and V_{L}-encoding DNA fragments can be operatively linked to another fragment encoding a flexible linker, for example, encoding the amino acid sequence (Gly₄-Ser)₃, such that the VH and VL sequences can be expressed as a contiguous single-chain protein, with the V_{L} and V_{H} domains joined by the flexible linker (see, for example, Bird et al., Science 242:423-426, 1988; Huston et al., Proc. Natl. Acad. Sci. USA 85:5879-5883, 1988; McCafferty et al., Nature 348:552-554, 1990; Kontermann and Dubel (Ed), Antibody Engineering, Vols. 1-2, 2^{nd} Ed., Springer Press, 2010; Harlow and Lane, Antibodies: A Laboratory Manual, 2^{nd}, Cold Spring Harbor Laboratory, New York, 2013,). Optionally, a cleavage site can be included in a linker, such as a furin cleavage site.

The nucleic acid encoding a V_{H} and/or the V_{L} optionally can encode an Fc domain. The Fc domain can be an IgA, IgM or IgG Fc domain. The Fc domain can be an optimized Fc domain, as described in U.S. Application Publication No. 20100/093979, incorporated herein by reference.

The single chain antibody may be monovalent, if only a single V_{H} and V_{L} are used, bivalent, if two V_{H} and V_{L} are used, or polyvalent, if more than two V_{H} and V_{L} are used. Bispecific or polyvalent antibodies may be generated that bind specifically to EBV gp42 and another antigen. The encoded V_{H} and V_{L} optionally can include a furin cleavage site between the V_{H} and V_{L} domains.

Numerous expression systems are available for expression of proteins including E. coli, other bacterial hosts, yeast, and various higher eukaryotic cells such as the COS, CHO, HeLa and myeloma cell lines.

One or more DNA sequences encoding the antibodies or conjugates can be expressed in vitro by DNA transfer into a suitable host cell. The cell may be prokaryotic or eukaryotic. The term also includes any progeny of the subject host cell. It is understood that all progeny may not be identical to the parental cell since there may be mutations that occur during replication. Methods of stable transfer, meaning that the foreign DNA is continuously maintained in the host, are known. Hybridomas expressing the antibodies of interest are also encompassed by this disclosure.

The expression of nucleic acids encoding the antibodies and antigen binding fragments described herein can be achieved by operably linking the DNA or cDNA to a promoter (which is either constitutive or inducible), followed by incorporation into an expression cassette. The promoter can be any promoter of interest, including a cytomegalovirus promoter and a human T cell lymphotropic virus promoter (HTLV)-1. Optionally, an enhancer, such as a cytomegalovirus enhancer, is included in the construct. The cassettes can be suitable for replication and integration in either prokaryotes or eukaryotes. Typical expression cassettes contain specific sequences useful for regulation of the expression of the DNA encoding the protein. For example, the expression cassettes can include appropriate promoters, enhancers, transcription and translation terminators, initiation sequences, a start codon (ATG) in front of a protein-encoding gene, splicing signal for introns, sequences for the maintenance of the correct reading frame of that gene to permit proper translation of mRNA, and stop codons. The vector can encode a selectable marker, such as a marker encoding drug resistance (for example, ampicillin or tetracycline resistance).

Modifications can be made to a nucleic acid encoding a polypeptide described herein without diminishing its biological activity. Some modifications can be made to facilitate the cloning, expression, or incorporation of the targeting molecule into a fusion protein. Such modifications include, for example, termination codons, a methionine added at the amino terminus to provide an initiation site, additional amino acids placed on either terminus to create conveniently located restriction sites, or additional amino acids (such as poly His) to aid in purification steps. In addition to recombinant methods, the immunoconjugates, effector moieties, and antibodies of the present disclosure can also be constructed in whole or in part using standard peptide synthesis.

Once expressed, the antibodies and conjugates can be purified according to standard procedures, including ammonium sulfate precipitation, affinity columns, column chromatography, and the like (see, generally, Simpson ed., Basic methods in Protein Purification and Analysis: A laboratory Manual, Cold Harbor Press, 2008). The antibodies and conjugates need not be 100% pure. Once purified, partially or to homogeneity as desired, if to be used therapeutically, the polypeptides should be substantially free of endotoxin.

Methods for expression of the antibodies and conjugates, and/or refolding to an appropriate active form, from mammalian cells, and bacteria such as E. coli have been described and are applicable to the antibodies disclosed herein (see, for example, Harlow and Lane, Antibodies: A Laboratory Manual, 2^{nd}, Cold Spring Harbor Laboratory, New York, 2013, Simpson ed., Basic methods in Protein Purification and Analysis: A laboratory Manual, Cold Harbor Press, 2008, and Ward et al., Nature 341:544, 1989.

In addition to recombinant methods, the antibodies and conjugates can also be constructed in whole or in part using standard peptide synthesis. Solid phase synthesis of the polypeptides can be accomplished by attaching the C-terminal amino acid of the sequence to an insoluble support followed by sequential addition of the remaining amino acids in the sequence. Techniques for solid phase synthesis are described by Barany & Merrifield, The Peptides: Analysis, Synthesis, Biology. Vol. 2: Special Methods in Peptide Synthesis, Part A. pp. 3-284; Merrifield et al., J. Am. Chem. Soc. 85:2149-2156, 1963, and Stewart et al., Solid Phase Peptide Synthesis, 2nd eel., Pierce Chem. Co., Rockford, Ill., 1984. Proteins of greater length may be synthesized by condensation of the amino and carboxyl termini of shorter fragments. Methods of forming peptide bonds by activation of a carboxyl terminal end (such as by the use of the coupling reagent N, N′-dicylohexylcarbodimide) can also be used.

### IX. Compositions and Methods of Use

A. Compositions

Compositions are provided that include one or more of the disclosed monoclonal antibodies that bind (for example specifically bind) EBV gp42 in a carrier. Compositions comprising multi-specific (such as bispecific or trispecific) antibodies, antibody conjugates and fusion proteins are also provided. The compositions are useful, for example, for the treatment or detection of an EBV infection. The compositions can be prepared in unit dosage forms for administration to a subject. The amount and timing of administration are at the discretion of the treating physician to achieve the desired purposes. The EBV gp42-specific antibody, bispecific antibody, fusion protein, antibody conjugate, or nucleic acid molecule encoding such molecules can be formulated for systemic or local administration. In one example, the EBV gp42-specific antibody, bispecific antibody, fusion protein, antibody conjugate, or nucleic acid molecule encoding such molecules, is formulated for parenteral administration, such as intravenous administration.

In some embodiments, the compositions includes an antibody, bispecific antibody, fusion protein, antibody conjugate, or nucleic acid molecule in at least 70%, at least 75%, at least 80%, at least 85%, at least 90%, at least 95%, at least 96%, at least 97%, at least 98% or at least 99% purity. In certain embodiments, the compositions contain less than 10%, less than 5%, less than 4%, less than 3%, less than 2%, less than 1% or less than 0.5% of macromolecular contaminants, such as other mammalian (for example, human) proteins.

The compositions for administration can include a solution of the EBV gp42-specific antibody, bispecific antibody, fusion protein, antibody conjugate, or nucleic acid molecule encoding such molecules, dissolved in a pharmaceutically acceptable carrier, such as an aqueous carrier. In some examples, such a composition is present in a container, such as a glass or plastic vial or syringe. A variety of aqueous carriers can be used, for example, buffered saline and the like. These solutions are sterile and generally free of undesirable matter. These compositions may be sterilized by conventional sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of antibody in these formulations can vary widely, and will be selected primarily based on fluid volumes, viscosities, body weight and the like in accordance with the particular mode of administration selected and the subject's needs.

A typical composition for intravenous administration includes about 0.01 to about 30 mg/kg of antibody or conjugate per subject per day. Actual methods for preparing administrable compositions will be known or apparent to those skilled in the art and are described in more detail in such publications as Remington's Pharmaceutical Science, 22th ed., Pharmaceutical Press, London, UK (2012). In some embodiments, the composition can be a liquid formulation including one or more antibodies or conjugates thereof in a concentration range from about 0.1 mg/ml to about 20 mg/ml, or from about 0.5 mg/ml to about 20 mg/ml, or from about 1 mg/ml to about 20 mg/ml, or from about 0.1 mg/ml to about 10 mg/ml, or from about 0.5 mg/ml to about 10 mg/ml, or from about 1 mg/ml to about 10 mg/ml.

The disclosed antibodies, bispecific antibodies, fusion proteins, antibody conjugates, and nucleic acids encoding such molecules, can be provided in lyophilized form and rehydrated with sterile water before administration, although they are also provided in sterile solutions of known concentration. The antibody solution, or an antigen binding fragment or a nucleic acid encoding such antibodies or antigen binding fragments, can then be added to an infusion bag containing 0.9% sodium chloride, USP, and administered according to standard protocols. Considerable experience is available in the art in the administration of antibody drugs, which have been marketed in the U.S. since the approval of RITUXAN ® in 1997. Antibodies, antigen binding fragments, conjugates, or a nucleic acid encoding such molecules, can be administered by slow infusion, rather than in an intravenous push or bolus. In one example, a higher loading dose is administered, with subsequent, maintenance doses being administered at a lower level. For example, an initial loading dose of 4 mg/kg may be infused over a period of some 90 minutes, followed by weekly maintenance doses for 4-8 weeks of 2 mg/kg infused over a 30 minute period if the previous dose was well tolerated.

Controlled-release parenteral formulations can be made as implants, oily injections, or as particulate systems. For a broad overview of protein delivery systems see, Banga, A. J., Therapeutic Peptides and Proteins: Formulation, Processing, and Delivery Systems, Technomic Publishing Company, Inc., Lancaster, PA, (1995). Particulate systems include microspheres, microparticles, microcapsules, nanocapsules, nanospheres, and nanoparticles. Microcapsules contain the therapeutic protein, such as a cytotoxin or a drug, as a central core. In microspheres the therapeutic is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly (see, for example, Kreuter, Colloidal Drug Delivery Systems, J. Kreuter, ed., Marcel Dekker, Inc., New York, NY, pp. 219-342 (1994); and Tice & Tabibi, Treatise on Controlled Drug Delivery, A. Kydonieus, ed., Marcel Dekker, Inc. New York, NY, pp. 315-339, (1992).

Polymers can be used for ion-controlled release of the antibody compositions disclosed herein. Various degradable and nondegradable polymeric matrices for use in controlled drug delivery have been described (Langer, Accounts Chem. Res. 26:537-542, 1993). For example, the block copolymer, polaxamer 407, exists as a viscous yet mobile liquid at low temperatures but forms a semisolid gel at body temperature. It is an effective vehicle for formulation and sustained delivery of recombinant interleukin-2 and urease (Johnston et al., Pharm. Res. 9:425-434, 1992; and Pec et al., J. Parent. Sci. Tech. 44(2):58-65, 1990). Alternatively, hydroxyapatite has been used as a microcarrier for controlled release of proteins (Ijntema et al., Int. J. Pharm. 112:215-224, 1994). In yet another aspect, liposomes are used for controlled release as well as drug targeting of the lipid-capsulated drug (Betageri et al., Liposome Drug Delivery Systems, Technomic Publishing Co., Inc., Lancaster, P A, 1993). Numerous additional systems for controlled delivery of therapeutic proteins have been described (see U.S. Pat. Nos. 5,055,303; 5,188,837; 4,235,871; 4,501,728; 4,837,028; 4,957,735; 5,019,369; 5,055,303; 5,514,670; 5,413,797; 5,268,164; 5,004,697; 4,902,505; 5,506,206; 5,271,961; 5,254,342 and 5,534,496).

B. Therapeutic and Prophylactic Methods

Disclosed herein are methods for the treatment or prophylaxis of an Epstein-Barr virus (EBV) infection in a subject. In some embodiments, the method includes administering to the subject a therapeutically effective amount of a gp42-specific monoclonal antibody, bispecific antibody, antibody conjugate, fusion protein, nucleic acid, vector or composition disclosed herein. In some examples, the method is a prophylactic method to prevent or inhibit infection by EBV. In specific non-limiting examples, the method is a prophylactic method and the subject is seronegative for EBV. In other specific non-limiting examples, the method is a prophylactic method and the subject is seropositive for EBV. In some examples, the method is a prophylactic method and the subject is immunocompromised or immunosuppressed. Alternatively, the patient may have a genetic disorder that makes the patient prone to develop life-threatening EBV disease. In specific examples, the subject has received an allograft, such as an organ transplant. In other examples, the method is a therapeutic method to treat a subject already infected with EBV. In specific non-limiting examples, the method includes selecting a subject who is infected with EBV.

In some embodiments, the method further includes administering to the subject one or more additional antibodies that specifically bind to an EBV protein, or one or more nucleic acid molecules encoding the additional antibodies. In some examples, the EBV protein is gp42, gp350, gH/gL or gB.

In therapeutic embodiments, the EBV infection in the subject does not need to be completely eliminated for the method to be effective. For example, the method can reduce or ameliorate EBV infection by a desired amount, for example by at least 10%, at least 20%, at least 50%, at least 60%, at least 70%, at least 80%, at least 90%, at least 95%, at least 98%, or 100% (elimination of detectable EBV infection or disease associated with EBV, such as infectious mononucleosis), as compared to EBV infection in the absence of the treatment.

In one non-limiting example, the method reduces viral titer in a subject with an EBV infection. For example, administration of a therapeutically effective amount of a disclosed EBV gp42-specific antibody, bispecific antibody, antibody conjugate, fusion protein, nucleic acid, vector or composition can reduce viral titer by at least 10%, at least 20%, at least 50%, at least 60%, at least 70%, at least 80%, at least 90%, at least 95%, at least 98%, or 100% (elimination of detectable EBV) in the subject. Methods of determining the EBV titer in the subject include, for example, obtaining a blood sample from the subject and assaying the sample for presence or activity of EBV.

In several embodiments, administration of a therapeutically effective amount of a disclosed EBV gp42-specific antibody, bispecific antibody, antibody conjugate, fusion protein, nucleic acid, vector or composition, results in a reduction in the establishment of EBV infection and/or reducing subsequent EBV-associated disease in a subject. A reduction in the establishment of EBV infection and/or a reduction in subsequent EBV-associated disease progression encompass any statistically significant reduction in EBV activity.

In several embodiments, the subject can be selected for treatment, for example, a subject at risk of EBV infection, or known to have an EBV infection. In some embodiments, a subject can be selected that is at risk of or known to have an infection with a particular type of EBV, such as EBV type 1 or EBV type 2. In some examples, the subject selected for treatment is one who has received a bone marrow transplant, an organ or tissue transplant, such as stem cell, hand, liver, heart, lung, kidney, pancreas, intestine, tendon, or skin transplant. In one example, the transplant is an allograft. In some examples, the subject selected for treatment is immunodeficient and may have a genetic disorder that increases the likelihood of developing life-threatening disease associated with EBV. In some examples, the subject selected for treatment is immunosuppressed. Thus, in some examples, a subject treated with one or more gp42 antibodies provided herein is one who has been treated with an immunosuppressant, such as one or more of a calcineurin inhibitor (e.g., cyclosporin, tacrolimus), corticosteroid (e.g., methylprednisolone, dexamethasone, prednisolone), a cytotoxic immunosuppressant (e.g., azathioprine, chlorambucil, cyclophosphamide, mercaptopurine, methotrexate), an immunosuppressant antibody (e.g., antithymocyte globulins, basiliximab, infliximab), a sirolimus derivative (e.g., everolimus, sirolimus), or another immunosuppressant such as mycophenolate. In one example, the subject is also treated with a calcineurin inhibitor such as Prograf (Tacrolimus) or Neoral (cyclosporine) with either Imuran (azathioprine) or CellCept (mycophenolate). In one example, the subject is also treated with a calcineurin inhibitor such as Prograf (Tacrolimus) or Neoral (cyclosporine), a corticosteroid, and either Imuran (azathioprine) or CellCept (mycophenolate). In one example, the subject is also treated with a calcineurin inhibitor such as Prograf (Tacrolimus) or Neoral (cyclosporine), a corticosteroid, either Imuran (azathioprine) or CellCept (mycophenolate), and an immunosuppressant antibody (antithymocyte globulin, basiliximab or daclizumab).

In several embodiments, a method of preventing or inhibiting EBV infection of a cell is provided. The method includes contacting the cell with an effective amount of a gp42-specific antibody, bispecific antibody, antibody conjugate, fusion protein or composition disclosed herein. For example, the cell can be incubated with the effective amount of the antibody or composition prior to or contemporaneous with incubation with the EBV. EBV infection of the cell does not need to be completely eliminated for the method to be effective. For example, a method can reduce EBV infection by a desired amount, for example by at least 10%, at least 20%, at least 50%, at least 60%, at least 70%, at least 80%, at least 90%, at least 95%, at least 98%, or 100% (elimination of detectable EBV infected cells), as compared to EBV infection in the absence of the treatment. In some embodiments, the cell is also contacted with an effective amount of an additional agent, such as an additional anti-viral agent. The cell can be in vivo or in vitro.

In some examples, a subject is administered a nucleic acid molecule (such as a DNA or modified RNA molecule) encoding the antibody, bispecific antibody, antibody conjugate or fusion protein, to provide in vivo antibody production, for example using the cellular machinery of the subject. Immunization by nucleic acid constructs is taught, for example, in U.S. Pat. Nos. 5,643,578, 5,593,972 and 5,817,637. U.S. Pat. No. 5,880,103 describes several methods of delivery of nucleic acids to an organism. One approach to administration of nucleic acids is direct administration with plasmid DNA, such as with a mammalian expression plasmid. The nucleotide sequence encoding the disclosed antibody can be placed under the control of a promoter to increase expression. The methods include liposomal delivery of the nucleic acids. In some embodiments, a disclosed antibody is expressed in a subject using the pVRC8400 vector (described in Barouch et al., J. Virol, 79:8828-8834, 2005, which is incorporated by reference herein).

The nucleic acid molecules encoding the disclosed antibodies or antigen binding fragments can be included in a viral vector, for example for expression of the antibody or antigen binding fragment in a host cell, or a subject (such as a subject with or at risk of EBV infection). A number of viral vectors have been constructed, that can be used to express the disclosed antibodies or conjugates thereof, such as a retroviral vector, an adenoviral vector, or an adeno-associated virus (AAV) vector. In several examples, the viral vector can be replication-competent. For example, the viral vector can have a mutation in the viral genome that does not inhibit viral replication in host cells. The viral vector also can be conditionally replication-competent. In other examples, the viral vector is replication-deficient in host cells.

In one embodiment, a nucleic acid encoding a disclosed antibody, bispecific antibody, antibody conjugate or fusion protein is introduced directly into cells. For example, the nucleic acid can be loaded onto gold microspheres by standard methods and introduced into the skin by a device such as Bio-Rad's HELIOS™ Gene Gun. The nucleic acids can be “naked,” consisting of plasmids under control of a strong promoter.

Typically, the nucleic acid is injected into muscle, although it can also be injected directly into other sites. Dosages for injection are usually around 0.5 μg/kg to about 50 mg/kg, and typically are about 0.005 mg/kg to about 5 mg/kg (see, e.g., U.S. Pat. No. 5,589,466).

Alternatively, using CRISPR-Cas, a nucleic acid encoding the antibody can be inserted into the genome, including stem cells and B lymphocytes, to express the disclosed antibodies or conjugates thereof.

1. Dosages

A therapeutically effective amount of an EBV gp42-specific antibody, bispecific antibody, antibody conjugate, fusion protein, or nucleic acid molecule (such as a vector) encoding such molecules, can depend upon the severity of the disease and/or infection and the general state of the patient's health. A therapeutically effective amount is that which provides either subjective relief of a symptom(s) or an objectively identifiable improvement as noted by the clinician or other qualified observer. The EBV gp42-specific antibody, bispecific antibody, antibody conjugate, fusion protein, or nucleic acid molecule encoding such molecules, can be administered in conjunction with another therapeutic agent, either simultaneously or sequentially.

Single or multiple administrations of a composition including a disclosed EBV gp42-specific antibody, bispecific antibody, antibody conjugate, fusion protein, or nucleic acid molecule encoding such molecules, can be administered depending on the dosage and frequency as required and tolerated by the patient. Compositions including the EBV gp42-specific antibody, bispecific antibody, antibody conjugate, fusion protein, or nucleic acid molecule encoding such molecules, should provide a sufficient quantity of at least one of the EBV gp42-specific antibodies, bispecific antibodies, antibody conjugates, fusion proteins, or nucleic acid molecules to effectively treat the patient. The dosage can be administered once, but may be applied periodically until either a therapeutic result is achieved or until side effects warrant discontinuation of therapy. In one example, a dose of the composition is infused for thirty minutes every other day. In this example, about one to about ten doses can be administered, such as three or six doses can be administered every other day. In a further example, a continuous infusion is administered for about five to about ten days. The subject can be treated at regular intervals, such as daily, weekly, or monthly, until a desired therapeutic result is achieved. Generally, the dose is sufficient to treat or ameliorate symptoms or signs of disease without producing unacceptable toxicity to the patient.

Data obtained from cell culture assays and animal studies can be used to formulate a range of dosage for use in humans. The dosage normally lies within a range of circulating concentrations that include the ED₅₀, with little or minimal toxicity. The dosage can vary within this range depending upon the dosage form employed and the route of administration utilized. The therapeutically effective dose can be determined from cell culture assays and animal studies.

In certain embodiments, the antibody, bispecific antibody, antibody conjugate or fusion protein that specifically binds EBV gp42, or a nucleic acid molecule or vector encoding such a molecule, can be administered at a dose in the range of from about 1 to about 100 mg/kg, such as about 5-50 mg/kg, about 25-75 mg/kg, or about 40-60 mg/kg. In some embodiments, the dosage can be administered at about 1, 2, 3, 4, 5, 6, 7, 8, 9, 10, 20, 30, 40, 50, 60, 70, 80, 90, 100, 150, 200, or 300 mg/kg, or other dose deemed appropriate by the treating physician. Further, the doses described herein can be administered according to the dosing frequency or frequency of administration described herein, including without limitation daily, every other day, 2 or 3 times per week, weekly, every 2 weeks, every 3 weeks, monthly, etc. In some embodiments, the dosage is administered daily beginning at the time of diagnosis with EBV and until EBV symptoms are alleviated. Additional treatments, including additional courses of therapy with a disclosed agent can be performed as needed.

2. Modes of Administration

The EBV gp42-specific antibody, bispecific antibody, antibody conjugate, fusion protein, nucleic acid molecule, vector or composition, as well as additional agents, can be administered to subjects in various ways, including local and systemic administration, such as, for example, by injection subcutaneously, intravenously, intra-arterially, intraperitoneally, intramuscularly, intradermally, or intrathecally. In an embodiment, a therapeutic agent is administered by a single subcutaneous, intravenous, intra-arterial, intraperitoneal, intramuscular, intradermal or intrathecal injection once a day. The therapeutic agent can also be administered by direct injection at or near the site of disease.

The therapeutic agent may also be administered orally in the form of microspheres, microcapsules, liposomes (uncharged or charged (such as cationic)), polymeric microparticles (such as polyamides, polylactide, polyglycolide, poly(lactide-glycolide)), microemulsions, and the like.

A further method of administration is by osmotic pump (for example, an Alzet pump) or mini-pump (for example, an Alzet mini-osmotic pump), which allows for controlled, continuous and/or slow-release delivery of the therapeutic agent or pharmaceutical composition over a pre-determined period. The osmotic pump or mini-pump can be implanted subcutaneously, or near a target site.

The therapeutic agent or compositions thereof can also be administered by other modes. The therapeutic agent can be administered as pharmaceutical formulations suitable for, for example, oral (including buccal and sub-lingual), rectal, nasal, topical, pulmonary, vaginal or parenteral (including intramuscular, intraarterial, intrathecal, subcutaneous and intravenous) administration, or in a form suitable for administration by inhalation or insufflation. Depending on the intended mode of administration, the pharmaceutical formulations can be in the form of solid, semi-solid or liquid dosage forms, such as tablets, suppositories, pills, capsules, powders, liquids, suspensions, emulsions, creams, ointments, lotions, and the like. The formulations can be provided in unit dosage form suitable for single administration of a precise dosage. The formulations comprise an effective amount of a therapeutic agent, and one or more pharmaceutically acceptable excipients, carriers and/or diluents, and optionally one or more other biologically active agents.

C. Methods for the Diagnosis or Detection of EBV

Also disclosed herein are methods for the detection of EBV and/or the diagnosis of EBV infection. In some embodiments, the method is a detection method that includes detecting EBV in a subject by contacting a biological sample from the subject with a gp42-specific monoclonal antibody disclosed herein under conditions sufficient to form an immune complex; and detecting the presence of the immune complex in the sample. The presence of the immune complex in the sample detects EBV in the subject. In other embodiments, the method is a diagnostic method that includes detecting EBV in a subject by contacting a biological sample from the subject with a gp42-specific monoclonal antibody disclosed herein under conditions sufficient to form an immune complex; and detecting the presence of the immune complex in the sample. The presence of the immune complex in the sample diagnosis the subject as having an EBV infection.

In some examples of the disclosed methods, the monoclonal antibody is directly labeled.

In other examples, the methods further include contacting a second antibody (a detection antibody) that specifically binds the monoclonal antibody with the sample; and detecting the binding of the second antibody. An increase in binding of the second antibody to the sample as compared to binding of the second antibody to a control sample detects EBV in the subject or diagnosis the subject as having an EBV infection. A second antibody is chosen that is able to specifically bind the specific species and class of the first antibody. For example, if the first antibody is a human IgG, then the secondary antibody may be an anti-human-IgG. Other molecules that can bind to antibodies include, without limitation, Protein A and Protein G, both of which are available commercially.

Suitable labels for the antibody or secondary antibody include various enzymes, prosthetic groups, fluorescent materials, luminescent materials, magnetic agents and radioactive materials. Non-limiting examples of suitable enzymes include horseradish peroxidase, alkaline phosphatase, beta-galactosidase, or acetylcholinesterase. Non-limiting examples of suitable prosthetic group complexes include streptavidin/biotin and avidin/biotin. Non-limiting examples of suitable fluorescent materials include umbelliferone, fluorescein, fluorescein isothiocyanate, rhodamine, dichlorotriazinylamine fluorescein, dansyl chloride or phycoerythrin. A non-limiting exemplary luminescent material is luminol; a non-limiting exemplary a magnetic agent is gadolinium, and non-limiting exemplary radioactive labels include ¹²⁵I, ¹³¹I, ³⁵S or ³H.

### X. Kits

Kits are also provided. For example, kits for treating a subject with an EBV infection, or for detecting EBV gp42 in a sample or in a subject. The kits can include a disclosed EBV gp42-specific monoclonal antibody or nucleic acid molecule encoding a gp42-specific monoclonal antibody, or compositions including such molecules, for example in a container, such as a glass, metal, or plastic container. More than one of the disclosed EBV gp42-specific antibody, conjugate, fusion protein or nucleic acid molecule/vector encoding such molecules, or compositions including such molecules can be included in the kit.

In one embodiment, the kit is a diagnostic kit and includes an immunoassay. Although the details of the immunoassays may vary with the particular format employed, the method of detecting EBV gp42 in a biological sample generally includes the steps of contacting the biological sample with an antibody which specifically reacts, under conditions sufficient to form an immune complex, to EBV gp42. The antibody is allowed to specifically bind under immunologically reactive conditions to form an immune complex, and the presence of the immune complex (bound antibody) is detected directly or indirectly. In some embodiments, the kits includes buffer, one or more detection reagents (such as a labeled secondary antibody or an enzyme) and/or instructional materials.

The kit can include a container and a label or package insert on or associated with the container. Suitable containers include, for example, bottles, vials, syringes, etc. The containers may be formed from a variety of materials such as glass or plastic. The container typically holds a composition including one or more of the disclosed antibodies, bi-specific antibodies, conjugates, fusion proteins, nucleic acid molecules, vectors or compositions. In several embodiments, the container may have a sterile access port (for example the container may be an intravenous solution bag or a vial having a stopper pierceable by a hypodermic injection needle). A label or package insert indicates that the composition is used for treating the particular condition.

The label or package insert typically will further include instructions for use of the antibodies, conjugates, fusion proteins, nucleic acid molecules, vectors or compositions included in the kit. The package insert typically includes instructions customarily included in commercial packages of therapeutic products that contain information about the indications, usage, dosage, administration, contraindications and/or warnings concerning the use of such therapeutic products. The instructional materials may be written, in an electronic form or may be visual. The kits may also include additional components to facilitate the particular application for which the kit is designed. Thus, for example, the kit may additionally contain means of detecting a label (such as enzyme substrates for enzymatic labels, filter sets to detect fluorescent labels, appropriate secondary labels such as a secondary antibody, or the like). The kits may additionally include buffers and other reagents routinely used for the practice of a particular method.

The following examples are provided to illustrate certain particular features and/or embodiments. These examples should not be construed to limit the disclosure to the particular features or embodiments described.

### EXAMPLES

### Example 1: Monoclonal Antibodies Specific for EBV Gp42

This example describes the generation and characterization of two EBV gp42-specific neutralizing monoclonal antibodies, referred to as A10 and 4C12.

### Non-Human Primate Antibody A10

A cynomolgus monkey was immunized with soluble EBV gH/gL/gp42. Single plasmablasts were isolated from PBMCs of the immunized monkey post vaccination. Heavy and light chain variable regions were amplified by PCR and cloned into an expression vector to generate a full-length rhesus antibody. The vector was transfected into 293F cells for expression of the antibody. The heavy and light chain variable regions were subsequently subcloned into a construct comprising the human constant region of IgG1. The resultant antibody is referred to as A10. The amino acid sequences of the VH and VL domains of A10 are set forth herein as SEQ ID NO: 2 and SEQ ID NO: 4, respectively.

### Mouse Antibody 4C12

Mice were immunized with soluble gp42. Splenic B cells from immunized mice were somatically fused to immortal myeloma cells to produce hybridomas (Precision Antibody, Columbia, MD). Supernatants from the hybridomas were screened, resulting in the selection of antibody 4C12. The heavy and light chain variable regions of 4C12 were cloned into a human IgG1 backbone. The amino acid sequences of the V_{H} and V_{L} domains of 4C12 are set forth herein as SEQ ID NO: 6 and SEQ ID NO: 8, respectively.

### Characterization of the A10 and 4C12 Antibodies

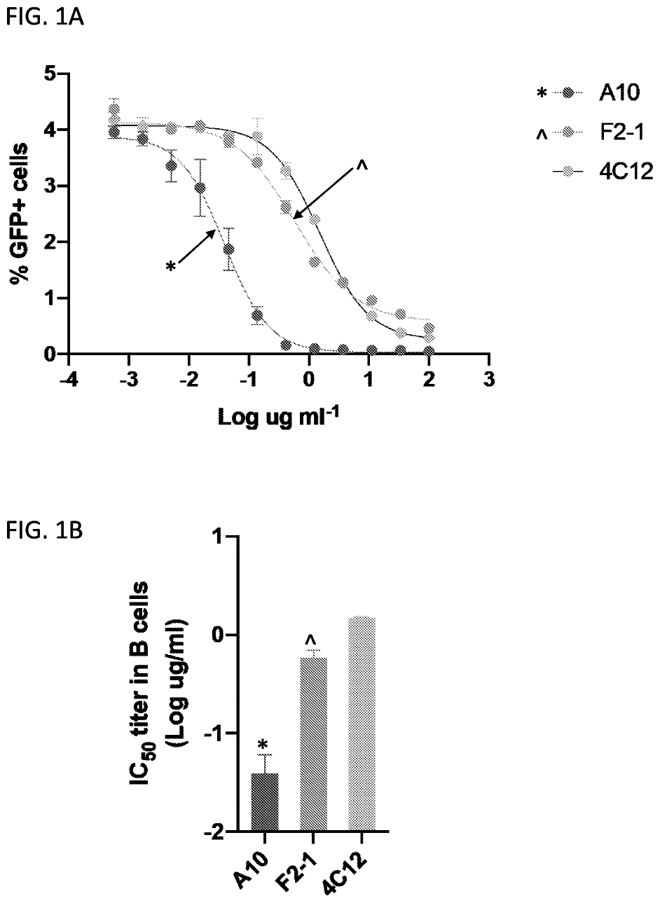

The ability of the A10 and 4C12 antibodies to neutralize EBV infection of B cells was compared to previously described murine anti-gp42 antibody F2-1. As shown in FIG. 1A, all three antibodies neutralized EBV infection to varying degrees, with antibody A10 being the most potent. Neutralization titers of the three antibodies are graphically depicted in FIG. 1B and quantified in Table 1 below. Antibody A10 was approximately 14-fold more potent than F2-1.

TABLE 1
IC₅₀ titers* of each mAb to neutralize EBV infection in B cells

| mAb | A10 | F2-1 | 4C12 |
|---|---|---|---|
| IC50 (μg/ml) | 0.03911 | 0.5843 | 1.504 |

*IC₅₀ titer = titer of antibody that gives 50% inhibition of infection

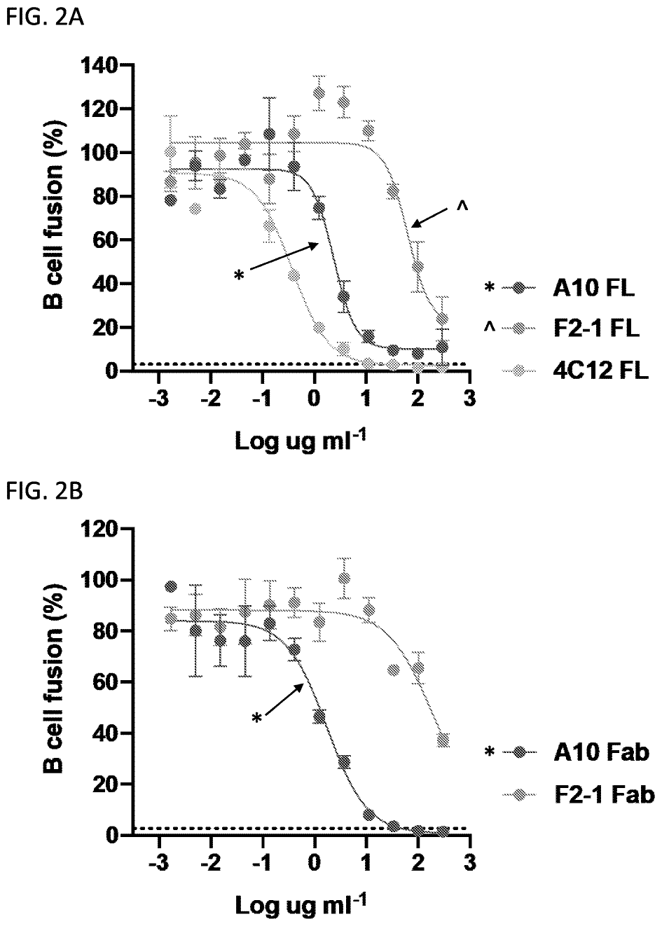

The three antibodies were also compared for their ability to block EBV glycoprotein-mediated B cell fusion. Both full-length mAbs (FIG. 2A) and Fabs (FIG. 2B) were evaluated. The percentage of fusion was defined as: (RLU [Rendla light units] of mAb Serum/RLU of Negative Serum)×100%. IC₅₀ titers of each mAb and Fab are listed in Table 2. 4C12 was the most effective antibody for blocking B cell fusion, followed by A10 and then F2-1. A10 and 4C12 mAbs were approximately 50-fold and 300-fold, respectively, more effective in blocking fusion than F2-1.

TABLE 2
IC₅₀ titers* of each mAb or Fab to block
glycoprotein-mediated B cell fusion

| mAb | A10 | F2-1 | 4C12 | A10 Fab | F2-1 Fab |
|---|---|---|---|---|---|
| IC50 (μg/ml) | 2.407 | 111.4 | 0.3770 | 1.726 | 187.0 |

*IC₅₀ titer = titer of antibody that gives 50% inhibition of glycoprotein-mediated cell fusion

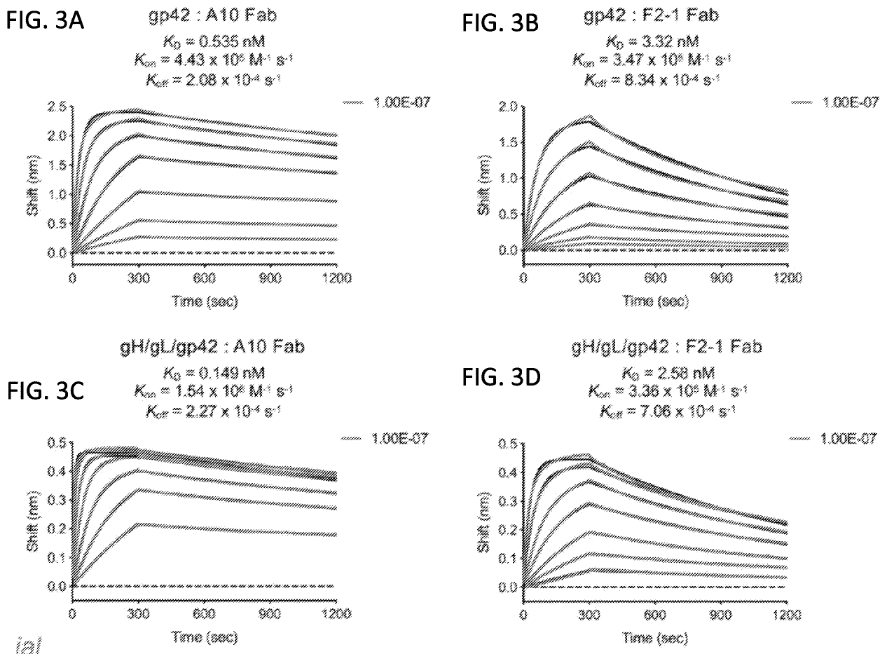

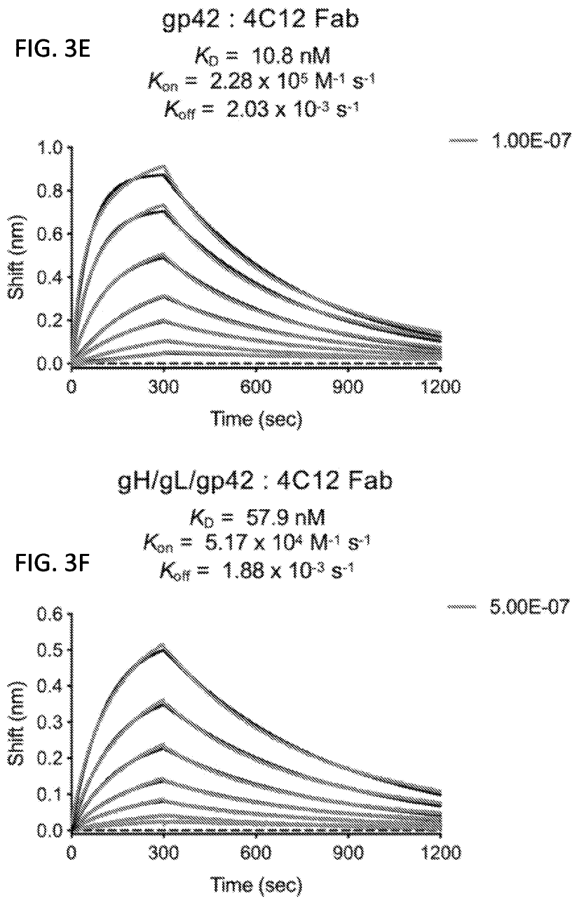

Binding affinity of the A10 and F2-1 antibodies was also determined. Biotinylated gp42 or gH/gL/gp42 proteins were immobilized on streptavidin biosensors (Fortébio). After briefly dipping in assay buffer (1% BSA in PBS), the biosensors were dipped in a 2-fold dilution series of Fab for 5 minutes. Biosensors were then dipped in assay buffer to allow Fabs to dissociate from gH/gL or gH/gL/gp42 for 15 minutes. All assay steps were performed at 30° C. with agitation set at 1,000 rpm in an Octet RED96e instrument (Fortébio). Data analysis and curve fitting were carried out using Octet analysis software (version 10.0). Experimental data were fitted with the binding equations describing a 1:1 interaction. Global analyses of the complete data sets assuming binding was reversible (full dissociation) were carried out using nonlinear least-squares fitting allowing a single set of binding parameters to be obtained simultaneously for all concentrations used in each experiment. The kinetics of F2-1 Fab and A10 Fab binding to gp42 and gH/gL/gh42 were measured and the results are shown in FIGS. 3A-3D. K_{d}, Kₒₙ and K_{dis} are listed in Table 3. The results demonstrated that the A10 Fab had a higher affinity for gp42 and gH/gL/gp42 than the F2-1 Fab.

TABLE 3
Binding affinity of A10 and F2-1 Fabs

| | gp42 + | gp42 + | gHgLgp42 + | gHgLgp42 + |
|---|---|---|---|---|
| | A10 Fab | F2-1 Fab | A10Fab | F2-1 Fab |
| K_{d} (M) | 5.35 × 10⁻¹⁰ | 3.32 × 10⁻⁹ | 1.49 × 10⁻¹⁰ | 2.58 × 10⁻⁹ |
| Kₒₙ (1/Ms) | 4.43 × 10⁵ | 3.47 × 10⁵ | 1.54 × 10⁶ | 3.36 × 10⁵ |
| K_{dis} (1/s) | 2.08 × 10⁻⁴ | 8.34 × 10⁻⁴ | 2.27 × 10⁻⁴ | 7.06 × 10⁻⁴ |

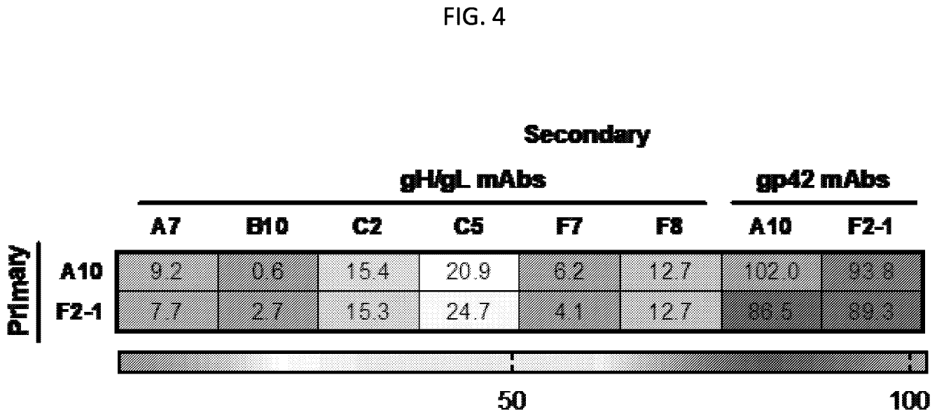

The gp42-specific monoclonal antibodies were further evaluated using competition assays. FIG. 4 shows a table of antibody cross-competition of EBV gp42 mAb A10 and F2-1 (primary antibodies) by gH/gL mAbs (A7, B10, C2, C5, F7, and F8) and gp42 mAbs (F2-1 and A10) (secondary antibodies). Antibody competition was measured by biolayer interferometry with recombinant gH/gL/gp42 protein. gH/gL/gp42 protein was bound to a biosensor and pre-saturated with primary antibodies and then the binding of secondary antibodies was measured to determine the extent that primary antibody can compete with secondary antibody for binding to gH/gL/gp42. The results demonstrated that gH/gL mAbs do not compete with gp42 mAbs for binding to gH/gL/gp42. Therefore, mAb A10 and F2-1 bind to gp42 and not to gH/gL.

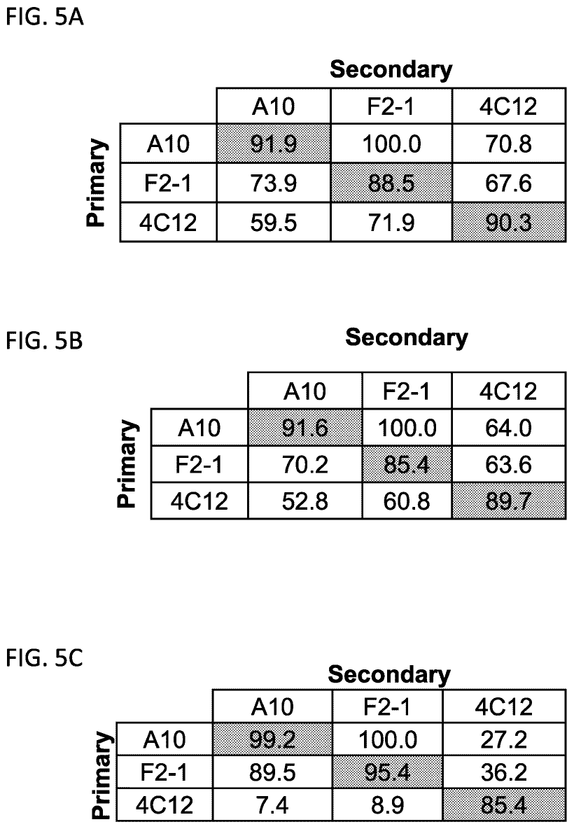

Shown in FIGS. 5A-5C are tables of antibody cross-competition of mAb A10 by F2-1 and 4C12. Antibody competition was measured by biolayer interferometry with recombinant EBV type I gp42 (FIG. 5A), EBV type II gp42 (FIG. 5B) or gH/gL/gp42 (FIG. 5C) proteins. There are two types of EBV-type I and type II-based on sequence differences. Both types can infect humans. Biosensors immobilized with ligands (gp42 or gH/gL/gp42 proteins) were pre-saturated with antibodies (primary) and then the binding of secondary antibodies was measured to determine the extent that primary antibody can compete with secondary antibody for binding to gp42 or gH/gL/gp42. The results showed that gp42 mAb A10 competed with F2-1 for binding to gp42 and gH/gL/gp42, indicating that A10 and F2-1 bind to similar sites on gp42 and gH/gL/gp42; mAb 4C-12 did not significantly compete with A10 or F2-1 for binding to gp42 and gH/gL/gp42, indicating that 4C12 binds to a different site on gp42 and gH/gL/gp42 than A10 or F2-1.

### Example 2: Antibody A10 Protects Against EBV Infection in a Humanized Mouse Model

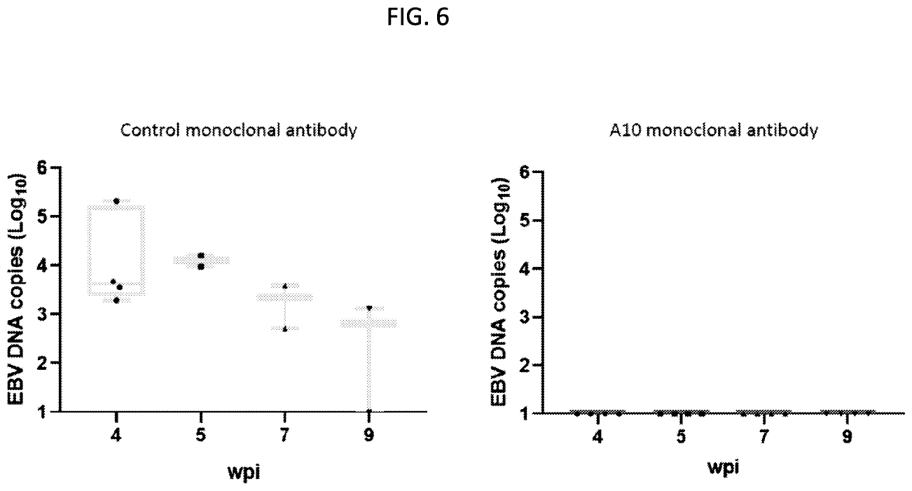

NOG female mice (NOG [NOD.Cg-Prkdc^{scid}Il2rg^{tm/Sug}/JicTac]) stably engrafted with human umbilical cord CD34+ hematopoietic stem cells for at least 12-16 weeks were given monoclonal antibody A10 (20 μg/g of mouse weight) intraperitoneally (i.p.) on day −1. The following day (day 0) the mice were given another dose of monoclonal antibody A10 i.p. and then EBV (one million cell culture infectious doses) intravenously. Additional doses of monoclonal antibody A10 were given on days 1, 4, 7 and 10. Animals were then bled on various weeks and the level of EBV in the blood was measured by extracting DNA from the blood and measuring the EBV DNA copy number by real-time PCR. As shown in FIG. 6, animals that received the A10 monoclonal antibody were protected from EBV viremia, while animals that received a control antibody developed viremia.

### Example 3. A10 Blocks Binding of Gp42 to HLA-DR and Human B Cells and Inhibits EBV Viremia

This example describes experiments to evaluate whether antibody A10 is capable of blocking gp42 from binding to its receptor HLA-DR and to human B cells.

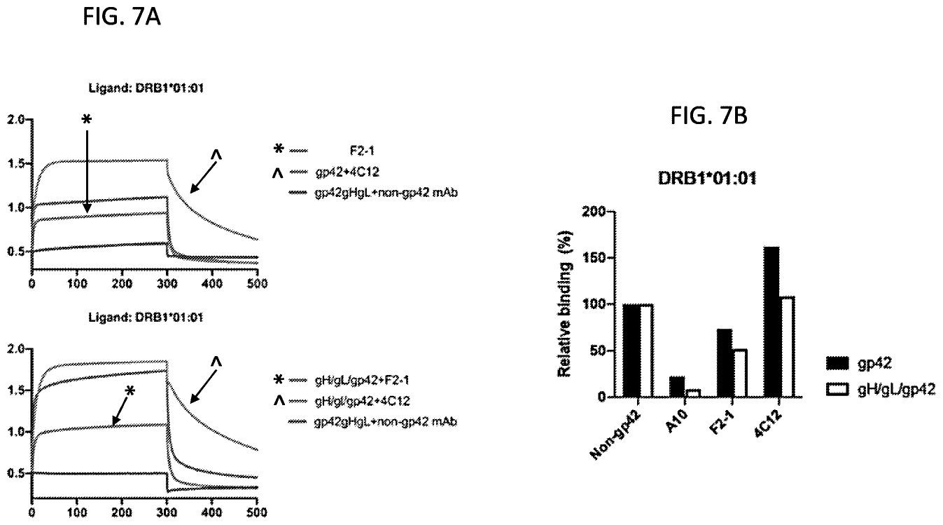

Ni-NTA biosensors were immobilized with gp42 or gH/gL/gp42 proteins and then dipped into a mixture of HLA-DR with either A10, F2-1, 4C12, or a control mAb for 300 seconds for association by biolayer interferometry, followed by dipping into buffer for dissociation. The results are shown in FIG. 7A. Relative binding of gp42 or gH/gL/gp42 to HLA-DR in the presence of mAbs was calculated by Response/Response_{controlAb}×100 (FIG. 7B). The results demonstrated that A10 significantly inhibits binding of gp42 and gH/gL/gp42 to HLA-DR.

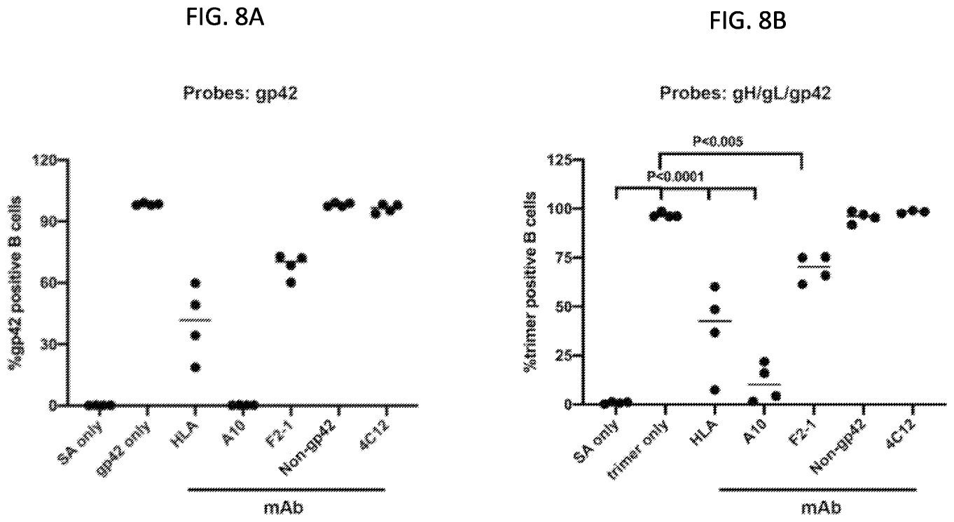

An additional study was performed to determine whether A10 is capable of blocking bindings of gp42 to human B cells. Fluorescent labeled gp42 or gH/gL/gp42 probes were mixed with anti-HLA, A10, F2-1, 4C12, or non-gp42 control mAbs followed by incubation with PBMCs from 4 individual healthy donors. The binding of probes on B cells was quantified by flow cytometry. The binding of probes only, without incubation of mAbs, was set as 100%. As shown in FIGS. 8A-8B, the A10 mAb significantly inhibited the ability of gp42 and gH/gL/gp42 to bind to human B cells.

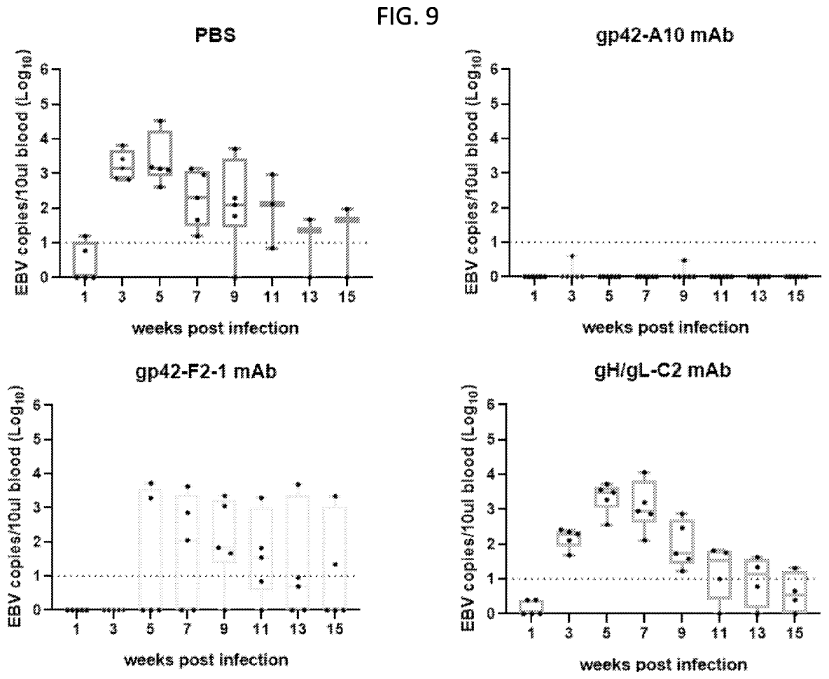

To evaluate whether blocking binding of gp42 to its receptor and to human B cells provides a therapeutic benefit, mAbs A10, 4C12 and F2-1 were tested for their ability to inhibit EBV infection. A10, 4C12 and F2-1 mAbs (0.5 mg per dose) were injected into humanized mice by I.P. injection on days −1, 0, 1, 4, 7, and 10. EBV (1×10⁶ Green Raji Units) was injected by I.V. injection on day 0. Blood was collected weekly and EBV copies were measured by real-time PCR. The results demonstrate that the A10 mAb provided the best protection from EBV viremia after challenge (FIG. 9).

### Example 4. A10 and Humanized A10-2 to A10-9 Neutralize EBV Infection of B Cells

This example describes B cell neutralization titers of the parental A10 and humanized A10 mAbs.

Eight humanized mAbs based on parental mAb A10 (designated as A10⁻² to A10⁻⁹) were synthesized. The humanized VH and VL domains were cloned into human IgG1 heavy chain and human kappa light chain expression vectors, respectively. M428L/N434S (“LS”) mutations were introduced into the heavy chain to extend half-life.

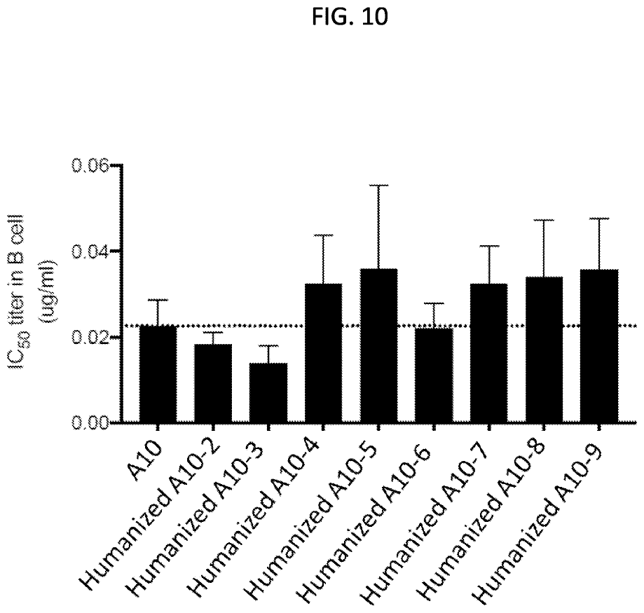

The humanized mAb data shown in FIG. 10 are IC₅₀ titers with standard errors of the mean based on 3 independent experiments. The results show that some the humanized A10 mAbs (for example, humanized A10-2 and humanized A10-3) neutralize EBV infection of B cells at lower concentrations than the parental A10 mAb.

In view of the many possible embodiments to which the principles of the disclosed subject matter may be applied, it should be recognized that the illustrated embodiments are only examples of the disclosure and should not be taken as limiting the scope of the disclosure. Rather, the scope of the disclosure is defined by the following claims. We therefore claim all that comes within the scope and spirit of these claims.

1. A monoclonal antibody that specifically binds Epstein-Barr virus (EBV) glycoprotein 42 (gp42), comprising a variable heavy (VH) domain and a variable light (VL) domain, wherein:
the VH domain comprises the complementarity determining region 1 (CDR1), CDR2 and CDR3 sequences of SEQ ID NO: 2 and the VL domain comprises the CDR1, CDR2 and CDR3 sequences of SEQ ID NO: 4 or SEQ ID NO: 11; or
the VH domain comprises the CDR1, CDR2 and CDR3 sequences of SEQ ID NO: 6 and the VL domain comprises the CDR1, CDR2 and CDR3 sequences of SEQ ID NO: 8.

2. The monoclonal antibody of claim 1, wherein the CDR sequences are defined using the Kabat, IMGT or Chothia numbering scheme.

3. The monoclonal antibody of claim 1, wherein:
the VH domain CDR1, CDR2 and CDR3 sequences respectively comprise residues 26-35, 53-61 and 100-109 of SEQ ID NO: 2 and the VL domain CDR1, CDR2 and CDR3 sequences respectively comprise residues 26-31, 49-51 and 88-99 of SEQ ID NO: 4 or SEQ ID NO: 11; or
the VH domain CDR1, CDR2 and CDR3 sequences respectively comprise residues 26-33, 51-58 and 97-109 of SEQ ID NO: 6 and the VL domain CDR1, CDR2 and CDR3 sequences respectively comprise residues 27-32, 50-52 and 89-97 of SEQ ID NO: 8.

4. The monoclonal antibody of claim 1, wherein:
the amino acid sequence of the VH domain is at least 90% identical to SEQ ID NO: 2, SEQ ID NO: 10, SEQ ID NO: 12, SEQ ID NO: 14, SEQ ID NO: 16, SEQ ID NO: 18, SEQ ID NO: 20, SEQ ID NO: 22 or SEQ ID NO: 24 and comprises the CDR1, CDR2 and CDR3 sequences of SEQ ID NO: 2; and
the amino acid sequence of the VL domain is at least 90% identical to SEQ ID NO: 4, SEQ ID NO: 11, SEQ ID NO: 13, SEQ ID NO: 15, SEQ ID NO: 17, SEQ ID NO: 19, SEQ ID NO: 21, SEQ ID NO: 23 or SEQ ID NO: 25 and comprises the CDR1, CDR2 and CDR3 sequences of SEQ ID NO: 4 or SEQ ID NO: 11.

5. The monoclonal antibody of claim 4, wherein:
the amino acid sequence of the VH domain comprises or consists of SEQ ID NO: 2, SEQ ID NO: 10, SEQ ID NO: 12, SEQ ID NO: 14, SEQ ID NO: 16, SEQ ID NO: 18, SEQ ID NO: 20, SEQ ID NO: 22 or SEQ ID NO: 24; and
the amino acid sequence of the VL domain comprises or consists of SEQ ID NO: 4, SEQ ID NO: 11, SEQ ID NO: 13, SEQ ID NO: 15, SEQ ID NO: 17, SEQ ID NO: 19, SEQ ID NO: 21, SEQ ID NO: 23 or SEQ ID NO: 25.

6. The monoclonal antibody of claim 1, wherein:
the amino acid sequence of the VH domain is at least 90% identical to SEQ ID NO: 6 and comprises the CDR1, CDR2 and CDR3 sequences of SEQ ID NO: 6; and
the amino acid sequence of the VL domain is at least 90% identical to SEQ ID NO: 8 and comprises the CDR1, CDR2 and CDR3 sequences of SEQ ID NO: 8.

7. The monoclonal antibody of claim 6, wherein:
the amino acid sequence of the VH domain comprises or consists of SEQ ID NO: 6; and
the amino acid sequence of the VL domain comprises or consists of SEQ ID NO: 8.

8. The monoclonal antibody of claim 1, wherein the antibody is an antigen-binding fragment selected from an Fab fragment, an Fab′ fragment, an F(ab)′₂ fragment, a single chain variable fragment (scFv) and a disulfide stabilized variable fragment (dsFv).

9. The monoclonal antibody of claim 1, wherein the antibody is an IgG molecule.

10. The monoclonal antibody of claim 9, wherein the IgG is an IgG1.

11. The monoclonal antibody of claim 1, wherein:
the monoclonal antibody is a mouse antibody;
the monoclonal antibody is a non-human primate antibody;
the monoclonal antibody is a humanized antibody; or
the monoclonal antibody is a chimeric antibody.

12-14. (canceled)

15. The monoclonal antibody of claim 1, comprising a constant region.

16. The monoclonal antibody of claim 15, wherein the constant region comprises at least one modification to increase half-life, stability and/or function of the monoclonal antibody.

17. A conjugate, comprising the monoclonal antibody of claim 1 linked to an effector molecule or a detectable label.

18. The conjugate of claim 17, wherein the detectable marker is a fluorescent, enzymatic, radioactive or nucleic acid label.

19. A bispecific antibody comprising the monoclonal antibody of claim 1 and a second monoclonal antibody or antigen-binding fragment thereof.

20. The bispecific antibody of claim 19, wherein the second monoclonal antibody binds a different epitope of gp42, or wherein the second monoclonal antibody binds an EBV protein other than gp42.

21. (canceled)

22. The bispecific antibody of claim 19, comprising:
a first monoclonal antibody comprising the VH domain CDR1, CDR2 and CDR3 sequences of SEQ ID NO: 2 and the VL domain CDR1, CDR2 and CDR3 sequences of SEQ ID NO: 4 or SEQ ID NO: 11; and
a second monoclonal antibody comprising the VH domain CDR1, CDR2 and CDR3 sequences of SEQ ID NO: 6 and the VL domain CDR1, CDR2 and CDR3 sequences of SEQ ID NO: 8.

23. A fusion protein comprising the monoclonal antibody of claim 1 and a heterologous protein.

24. An isolated nucleic acid molecule encoding the monoclonal antibody of claim 1.

25. The isolated nucleic acid molecule of claim 24, comprising:
the nucleotide sequence of SEQ ID NO: 1, or a degenerate variant thereof;
the nucleotide sequence of SEQ ID NO: 3, or a degenerate variant thereof;
the nucleotide sequences of SEQ ID NO: 1 and SEQ ID NO: 3, or degenerate variants thereof;
the nucleotide sequence of SEQ ID NO: 5, or a degenerate variant thereof;
the nucleotide sequence of SEQ ID NO: 7, or a degenerate variant thereof; or
the nucleotide sequences of SEQ ID NO: 5 and SEQ ID NO: 7, or degenerate variants thereof.

26. (canceled)

27. The isolated nucleic acid molecule of claim 24, operably linked to a promoter.

28. A vector comprising the nucleic acid molecule of claim 24.

29. A composition, comprising the monoclonal antibody of claim 1, and a pharmaceutically acceptable carrier.

30. A method of detecting Epstein-Barr virus (EBV) in a subject, comprising:
contacting a biological sample from the subject with the monoclonal antibody of claim 1 under conditions sufficient to form an immune complex; and
detecting the presence of the immune complex in the sample, wherein the presence of the immune complex in the sample detects EBV in the subject.

31. A method for the treatment or prophylaxis of an Epstein-Barr virus (EBV) infection in a subject, comprising administering to the subject a therapeutically effective amount of the monoclonal antibody of claim 1, thereby preventing or treating the EBV infection.

32-33. (canceled)

34. The method of claim 31, wherein:
the method is a prophylactic method or a therapeutic method and the subject is seronegative for EBV;
the method is a prophylactic method or a therapeutic method and the subject is seropositive for EBV;
the method is a prophylactic method or a therapeutic method and the subject is immunosuppressed;
the method is a prophylactic method or a therapeutic method and the subject is immunocompromised;
the subject has been diagnosed with an EBV infection; and/or
the subject has received an organ, bone marrow or tissue transplant.

35-39. (canceled)

40. A method of preventing or inhibiting EBV-associated lymphoproliferative disease in a subject, comprising administering to the subject a therapeutically effective amount of the monoclonal antibody of claim 1, thereby preventing or inhibiting EBV-associated lymphoproliferative disease.

41. The method of claim 40, wherein:
the subject is immunocompromised or immunosuppressed; and/or
the subject has received an organ, bone marrow or tissue transplant.

42. (canceled)

43. A kit comprising:
(i) the monoclonal antibody of claim 1; and
(ii) a buffer, one or more detection reagents, and/or instructional materials.


SEQUENCE LISTING (25 sequences)

SEQ ID NO: 1 - DNA, 360 bp, Artificial Sequence
  Synthetic polynucleotide
caggtgcagctgcaggagtcaggtccaggactggtgaagccctcacagaccctgtcactcacctgtgccatctccggggacagtgtctctagcaacagtgctacttggaactggatcaggcagtcgccatcgagaggccttgagtggctgggaaggacatactacaggtccaagtggtattatgattatgcacaatctgtgcaaaatcgaataagcatcaacccagacacatccaagaaccagttctccctgcagctgaactctgtgacccccgaggacatggccgtgtattactgtgcaagaggggattttgatgcttttgatttctggggccaagggctcagggtcaccgtctcttca

SEQ ID NO: 2 - protein, 120 aa, Artificial Sequence
  Synthetic polypeptide
QVQLQESGPGLVKPSQTLSLTCAISGDSVSSNSATWNWIRQSPSRGLEWLGRTYYRSKWYYDYAQSVQNRISINPDTSKNQFSLQLNSVTPEDMAVYYCARGDFDAFDFWGQGLRVTVSS

SEQ ID NO: 3 - DNA, 321 bp, Artificial Sequence
  Synthetic polynucleotide
tcgtatgagctgactcagccaccctcagtgtcagcggcctcaggacagacggccaggatcacctgtgggggagacaacattggaagtaaaaatgtgcagtggtaccagcagaagccagcgcaggcccctgtgctggtcatctatgctgatagtaaacggccctcaggggtccctgagcgattctctggctccaactcagggaacacggccaccctgaccatcagcggggtcgaggccggggatgaggctgactattactgtcaggtgtgggacaggagtagtaacatcttcggtgttgggacccggctcaccgtcctaggt

SEQ ID NO: 4 - protein, 107 aa, Artificial Sequence
  Synthetic polypeptide
SYELTQPPSVSAASGQTARITCGGDNIGSKNVQWYQQKPAQAPVLVIYADSKRPSGVPERFSGSNSGNTATLTISGVEAGDEADYYCQVWDRSSNIFGVGTRLTVLG

SEQ ID NO: 5 - DNA, 360 bp, Artificial Sequence
  Synthetic polynucleotide
gaggtccagcttcagcagtcaggacctgagctggtgaaacctggggcctcagtgaagatatcctgcaaggcttctggatacacattcactgactacaacatgcactgggtgaagcagagccatggaaagaaccttgagtggattggatttatttatctttacaatggtggtactggctacaaccagaacttcaagagcaaggccacattgactgtagacaattcctccagcacagcctacatggagctccgcagcctgacatctgaggactctgcagtcttttactgtgcaagggactactatggtaacccctatgctatggactactggggtcaaggaacctcagtcaccgtctcctca

SEQ ID NO: 6 - protein, 120 aa, Artificial Sequence
  Synthetic polypeptide
EVQLQQSGPELVKPGASVKISCKASGYTFTDYNMHWVKQSHGKNLEWIGFIYLYNGGTGYNQNFKSKATLTVDNSSSTAYMELRSLTSEDSAVFYCARDYYGNPYAMDYWGQGTSVTVSS

SEQ ID NO: 7 - DNA, 321 bp, Artificial Sequence
  Synthetic polynucleotide
gatatccagatgacacagactacatcctccctgtctgcctctctgggagacagagtcaccatcagttgcagggcaagtcaggacattagcaattatttaaactggtatcagcagaaaccagatggaactgttaaactcctaatctactacacatcaagattacgctcaggagtcccatcaaggttcagtggcagtgggtctggaacagattattctctcaccattaacaacctggagcaagaagatattgccacttacttttgccaacagggtaatacgcttccattcacgttcggctcggggacaaagttggaaatcaaa

SEQ ID NO: 8 - protein, 107 aa, Artificial Sequence
  Synthetic polypeptide
DIQMTQTTSSLSASLGDRVTISCRASQDISNYLNWYQQKPDGTVKLLIYYTSRLRSGVPSRFSGSGSGTDYSLTINNLEQEDIATYFCQQGNTLPFTFGSGTKLEIK

SEQ ID NO: 9 - protein, 223 aa, Human herpesvirus 4
MVSFKQVRVPLFTAIALVIVLLLAYFLPPRVRGGGRVAAAAITWVPKPNVEVWPVDPPPPVNFNKTAEQEYGDKEVKLPHWTPTLHTFQVPQNYTKANCTYCNTREYTFSYKGCCFYFTKKKHTWNGCFQACAELYPCTYFYGPTPDILPVVTRNLNAIESLWVGVYRVGEGNWTSLDGGTFKVYQIFGSHCTYVSKFSTVPVSHHECSFLKPCLCVSQRSNS

SEQ ID NO: 10 - protein, 120 aa, Artificial Sequence
  Synthetic polypeptide
QVQLQESGPGLVKPSQTLSLTCTISGDSVSSNSATWNWIRQPPGKGLEWLGRTYYRSKWYYDYAQSLKSRVTISPDTSKNQFSLKLSSVTAADTAVYYCARGDFDAFDFWGQGTLVTVSS

SEQ ID NO: 11 - protein, 107 aa, Artificial Sequence
  Synthetic polypeptide
SYELMQPPSVSVSPGQTARITCSGDNIGSKNVQWYQQKPGQAPVLVIYADSKRPSGIPERFSGSNSGNTATLTISGVQAEDEADYYCQVWDRSSNIFGGGTKLTVLG

SEQ ID NO: 12 - protein, 120 aa, Artificial Sequence; identical to SEQ ID NO: 10
  Synthetic polypeptide

SEQ ID NO: 13 - protein, 107 aa, Artificial Sequence
  Synthetic polypeptide
SYELTQPPSVSVSPGQTARITCSGDNIGSKNVQWYQQKSGQAPVLVIYADSKRPSGIPERFSGSNSGNMATLTISGAQVEDEDDYYCQVWDRSSNIFGGGTKLTVLG

SEQ ID NO: 14 - protein, 120 aa, Artificial Sequence; identical to SEQ ID NO: 10
  Synthetic polypeptide

SEQ ID NO: 15 - protein, 107 aa, Artificial Sequence
  Synthetic polypeptide
SYVLTQPPSVSAAPGQKVTISCSGDNIGSKNVQWYQQLPGTAPKLLIYADSKRPSGIPDRFSGSNSGNSATLGITGLQTGDEADYYCQVWDRSSNIFGGGTKLTVLG

SEQ ID NO: 16 - protein, 120 aa, Artificial Sequence; identical to SEQ ID NO: 10
  Synthetic polypeptide

SEQ ID NO: 17 - protein, 107 aa, Artificial Sequence
  Synthetic polypeptide
QYVLTQPPSVSAAPGQKVTISCSGDNIGSKNVQWYQQLPGTAPKLLIYADSKRPSGIPDRFSGSNSGNSATLGITGLQTGDEADYYCQVWDRSSNIFGGGTKLTVLG

SEQ ID NO: 18 - protein, 120 aa, Artificial Sequence
  Synthetic polypeptide
QVQLQESGPGLVKPSQTLSLTCTISGDSVSSNSATWNWIRQPPGKGLEWIGRTYYRSKWYYDYAQSLKSRVTISPDTSKNQFSLKLSSVTAADTAVYYCARGDFDAFDFWGQGTLVTVSS

SEQ ID NO: 19 - protein, 107 aa, Artificial Sequence; identical to SEQ ID NO: 11
  Synthetic polypeptide

SEQ ID NO: 20 - protein, 120 aa, Artificial Sequence; identical to SEQ ID NO: 18
  Synthetic polypeptide

SEQ ID NO: 21 - protein, 107 aa, Artificial Sequence; identical to SEQ ID NO: 13
  Synthetic polypeptide

SEQ ID NO: 22 - protein, 120 aa, Artificial Sequence; identical to SEQ ID NO: 18
  Synthetic polypeptide

SEQ ID NO: 23 - protein, 107 aa, Artificial Sequence; identical to SEQ ID NO: 15
  Synthetic polypeptide

SEQ ID NO: 24 - protein, 120 aa, Artificial Sequence; identical to SEQ ID NO: 18
  Synthetic polypeptide

SEQ ID NO: 25 - protein, 107 aa, Artificial Sequence; identical to SEQ ID NO: 17
  Synthetic polypeptide